(19) United States
(12) Patent Application Publication
(10) Pub. No.: US 2025/0026843 A1
(43) Pub. Date: Jan. 23, 2025

(54) BISPECIFIC ANTIBODY AGAINST CD40 AND CLDN18.2 AND APPLICATION THEREOF

(71) Applicant: ANHUI BIOX-VISION BIOLOGICAL TECHNOLOGY CO., LTD., Anhui (CN)

(72) Inventors: Guojun LANG, Shanghai (CN); Wenhai ZHANG, Shanghai (CN); Guoxing WANG, Shanghai (CN); Run YAN, Shanghai (CN); Zhen ZHANG, Shanghai (CN); Yuhao HU, Shanghai (CN); Peipei LIU, Shanghai (CN); Li FAN, Shanghai (CN); Yongcong TAN, Shanghai (CN)

(73) Assignee: ANHUI BIOX-VISION BIOLOGICAL TECHNOLOGY CO., LTD., Anhui (CN)

(21) Appl. No.: 18/715,119

(22) PCT Filed: Dec. 28, 2022
(86) PCT No.: PCT/CN2022/142771

§ 371 Date: May 31, 2024

(30) Foreign Application Priority Data
Dec. 30, 2021 (CN) 202111655014.3

(51) Int. Cl. C07K 16/28; A61K 39/00; A61P 35/00

(52) U.S. Cl. CPC C07K 16/2878; A61P 35/00; C07K 16/28; A61K 2039/505; C07K 2317/31; C07K 2317/565; C07K 2317/569; C07K 2317/92

(57) ABSTRACT

A bispecific antibody targeting both CD40 and CLDN18.2, a pharmaceutical composition comprising same, an application thereof for treating related diseases, and a treatment method are provided. The bispecific antibody targeting both CD40 and CLDN18.2 comprises (i) an anti-CD40 antibody or a fragment thereof and (ii) an anti-CLDN18.2 antibody or a fragment thereof.

### TECHNICAL BACKGROUND

CD40 is a co-stimulatory protein with a molecular weight of 50 kD located on a cell membrane surface, belongs to the tumor necrosis factor receptor (TNFR) family, is constitutively expressed in antigen-presenting cells (including dendritic cells, B cells, monocytes, and macrophages), and is also expressed in various other cells, such as endothelial cells, fibroblasts, and thymic epithelial cells. In addition, CD40 is also found to be expressed in various tumor cells (for example, chronic lymphocytic leukemia, multiple myeloma, renal cancer, lung cancer, etc.). CD40 forms a trimer on the cell surface, and a corresponding ligand CD40L (that is, CD154) is mainly expressed on activated T cell surfaces. The interactive effect between CD40 and CD40L is a co-stimulatory signal for T cell activation. The binding of CD40L to CD40 on T cells can activate multiple pathways, including NF-κB (nuclear factor KB) signaling pathway.

Although agonistic antibodies targeting CD40, such as selicrelumab, dacetuzumab, and APX005, show clinical activity in various indications in clinical trials, the agonistic antibodies are related to adverse events of dose-limiting toxicity (Hassan S B et al., Anti-CD40-mediated cancer immunotherapy: an update of recent and ongoing clinical trials. Immunopharmacol Immunotoxicol 2014; 36:96-104). Currently, the main adverse event is hepatotoxicity. The agonistic antibodies non-specifically activate immune cells expressing CD40 on surfaces hepatocytes, leading to hepatotoxicity and reducing the therapeutic index.

The human CLDN18 gene has two different exons 1, which undergo alternative splicing after transcription to ultimately produce two protein isoforms CLDN18.1 and CLDN18.2 that have different sequences only at the N-terminus. The two CLDN18 protein isoforms are both composed of 261 amino acids and both have four transmembrane domains, but the two are distributed in different tissues. CLDN18.1 is mainly expressed in lung tissue, while CLDN18.2 is only expressed in differentiated gastric mucosal epithelial cells and not in gastric stem cells (Sahin, Ugur, et al. “Claudin-18 splice variant 2 is a pan-cancer target suitable for therapeutic antibody development.” Clinical Cancer Research 14.23 (2008): 7624-7634.). CLDN18.2 is highly expressed in various tumor tissues, such as non-small cell lung cancer (25%), gastric cancer (70%), pancreatic cancer (50%), and esophageal cancer (30%), but is almost not expressed in normal tissues (Kumar, V., et al. (2018). “Emerging Therapies in the Management of Advanced-Stage Gastric Cancer.” Front Pharmacol 9:404). Due to the differential expression in tumor cells and normal tissues, CLDN18.2 has become a very potential target for anti-tumor drugs.

Therefore, there is an urgent need to develop a bispecific antibody targeting CLDN18.2 and CD40 to achieve specific killing of tumor cells without causing adverse events such as hepatotoxicity, providing more possibilities for cancer treatment.

### SUMMARY

The present invention discloses a novel bispecific antibody targeting both CD40 and CLDN18.2, a polynucleotide encoding the bispecific antibody, a vector including the polynucleotide, a host cell including the polynucleotide or the vector, a method for treating a disease related to CD40 and/or CLDN18.2 by adopting the bispecific antibody, and a use of the bispecific antibody in individual treatment, prevention, and/or diagnosis of the disease related to CD40 and/or CLDN18.2. The bispecific antibody can implement high-intensity activation of immune cells specifically at a lesion site, while implementing low agonistic activity in places such as hepatocytes that cannot form cross-linking effects, thereby implementing specific killing of tumor cells without causing adverse events such as hepatotoxicity.

In a first aspect, the present invention provides a bispecific antibody specifically binding to CD40 and CLDN18.2 (an anti-CD40×CLDN18.2 bispecific antibody), which includes (i) an anti-CD40 antibody or a fragment thereof and (ii) an anti-CLDN18.2 antibody or a fragment thereof. In an embodiment, the anti-CLDN18.2 antibody or the fragment thereof is a single domain antibody (VHH).

In an embodiment, the present invention provides an anti-CD40×CLDN18.2 bispecific antibody, which includes:
(i) a heavy chain with a structure VH-CH1-Fc-VHH, and
(ii) a light chain with a structure VL-CL,
wherein VH and VL are combined to form a first antigen binding part specifically binding to CD40, and VHH forms a second antigen binding part specifically binding to CLDN18.2, optionally, VHH is connected to Fc through a linker.

In an embodiment, the present invention provides an anti-CD40×CLDN18.2 bispecific antibody, which includes:
(i) a heavy chain with a structure VH-CH1-Fc-VHH, and
(ii) a light chain with a structure VL-CL,
wherein
the VH includes 3 heavy chain CDRs contained in SEQ ID NO:11, the VL includes 3 light chain CDRs contained in SEQ ID NO: 10, and the VHH includes 3 CDRs contained in SEQ ID NO: 18;
the VH includes 3 heavy chain CDRs contained in SEQ ID NO:12, the VL includes 3 light chain CDRs contained in SEQ ID NO: 10, and the VHH includes 3 CDRs contained in SEQ ID NO: 18;
the VH includes 3 heavy chain CDRs contained in SEQ ID NO:13, the VL includes 3 light chain CDRs contained in SEQ ID NO: 10, and the VHH includes 3 CDRs contained in SEQ ID NO: 18; or
the VH includes 3 heavy chain CDRs contained in SEQ ID NO: 14, the VL includes 3 light chain CDRs contained in SEQ ID NO: 10, and the VHH includes 3 CDRs contained in SEQ ID NO: 18.

In another embodiment, the present invention provides an anti-CD40×CLDN18.2 bispecific antibody, which includes:
(i) a heavy chain with a structure VH-CH1-Fc-VHH, and
(ii) a light chain with a structure VL-CL,
wherein
the VH includes 3 heavy chain CDRs shown in SEQ ID NO:4, SEQ ID NO:5, and SEQ ID NO: 6, the VL includes 3 light chain CDRs shown in SEQ ID NO:1, SEQ ID NO:2, and SEQ ID NO: 3, and the VHH includes 3 CDRs shown in SEQ ID NO: 15, SEQ ID NO: 16, and SEQ ID NO: 17;
the VH includes 3 heavy chain CDRs shown in SEQ ID NO:4, SEQ ID NO:5, and SEQ ID NO: 7, the VL includes 3 light chain CDRs shown in SEQ ID NO:1, SEQ ID NO:2, and SEQ ID NO: 3, and the VHH includes 3 CDRs shown in SEQ ID NO:15, SEQ ID NO: 16, and SEQ ID NO: 17;
the VH includes 3 heavy chain CDRs shown in SEQ ID NO:4, SEQ ID NO:5, and SEQ ID NO: 8, the VL includes 3 light chain CDRs shown in SEQ ID NO:1, SEQ ID NO:2, and SEQ ID NO: 3, and the VHH includes 3 CDRs shown in SEQ ID NO: 15, SEQ ID NO: 16, and SEQ ID NO: 17; or
the VH includes 3 heavy chain CDRs shown in SEQ ID NO:4, SEQ ID NO:5, and SEQ ID NO: 9, the VL includes 3 light chain CDRs shown in SEQ ID NO: 1, SEQ ID NO:2, and SEQ ID NO: 3, and the VHH includes 3 CDRs shown in SEQ ID NO: 15, SEQ ID NO: 16, and SEQ ID NO: 17.

In yet another embodiment, the present invention provides an anti-CD40×CLDN18.2 bispecific antibody, which includes:
(i) a heavy chain with a structure VH-CH1-Fc-VHH, and
(ii) a light chain with a structure VL-CL,
wherein
the VH includes 3 heavy chain CDRs contained in SEQ ID NO: 11 and a sequence having at least 90%, 91%, 92%, 93%, 94%, 95%, 96%, 97%, 98%, 99%, or more sequence identity with SEQ ID NO:11, the VL includes 3 light chain CDRs contained in SEQ ID NO: 10 and a sequence having at least 90%, 91%, 92%, 93%, 94%, 95%, 96%, 97%, 98%, 99%, or more sequence identity with SEQ ID NO: 10, and the VHH includes 3 CDRs contained in SEQ ID NO: 18 and a sequence having at least 90%, 91%, 92%, 93%, 94%, 95%, 96%, 97%, 98%, 99%, or more sequence identity with SEQ ID NO:18;
the VH includes 3 heavy chain CDRs contained in SEQ ID NO: 12 and a sequence having at least 90%, 91%, 92%, 93%, 94%, 95%, 96%, 97%, 98%, 99%, or more sequence identity with SEQ ID NO:12, the VL includes 3 light chain CDRs contained in SEQ ID NO:10 and a sequence having at least 90%, 91%, 92%, 93%, 94%, 95%, 96%, 97%, 98%, 99%, or more sequence identity with SEQ ID NO: 10, and the VHH includes 3 CDRs contained in SEQ ID NO:18 and a sequence having at least 90%, 91%, 92%, 93%, 94%, 95%, 96%, 97%, 98%, 99%, or more sequence identity with SEQ ID NO:18;
the VH includes 3 heavy chain CDRs contained in SEQ ID NO: 13 and a sequence having at least 90%, 91%, 92%, 93%, 94%, 95%, 96%, 97%, 98%, 99%, or more sequence identity with SEQ ID NO:13, the VL includes 3 light chain CDRs contained in SEQ ID NO: 10 and a sequence having at least 90%, 91%, 92%, 93%, 94%, 95%, 96%, 97%, 98%, 99%, or more sequence identity with SEQ ID NO: 10, and the VHH includes 3 CDRs contained in SEQ ID NO: 18 and a sequence having at least 90%, 91%, 92%, 93%, 94%, 95%, 96%, 97%, 98%, 99%, or more sequence identity with SEQ ID NO: 18; or
the VH includes 3 heavy chain CDRs contained in SEQ ID NO: 14 and a sequence having at least 90%, 91%, 92%, 93%, 94%, 95%, 96%, 97%, 98%, 99%, or more sequence identity with SEQ ID NO:14, the VL includes 3 light chain CDRs contained in SEQ ID NO: 10 and a sequence having at least 90%, 91%, 92%, 93%, 94%, 95%, 96%, 97%, 98%, 99%, or more sequence identity with SEQ ID NO: 10, and the VHH includes 3 CDRs contained in SEQ ID NO: 18 and a sequence having at least 90%, 91%, 92%, 93%, 94%, 95%, 96%, 97%, 98%, 99%, or more sequence identity with SEQ ID NO: 18.

In an embodiment, the present invention provides an anti-CD40×CLDN18.2 bispecific antibody, which includes:
(i) a heavy chain with a structure VH-CH1-Fc-VHH, and
(ii) a light chain with a structure VL-CL,
wherein
the VH includes a sequence shown in SEQ ID NO: 11, the VL includes a sequence shown in SEQ ID NO:10, and the VHH includes a sequence shown in SEQ ID NO:18;
the VH includes a sequence shown in SEQ ID NO: 12, the VL includes a sequence shown in SEQ ID NO:10, and the VHH includes a sequence shown in SEQ ID NO:18;
the VH includes a sequence shown in SEQ ID NO: 13, the VL includes a sequence shown in SEQ ID NO:10, and the VHH includes a sequence shown in SEQ ID NO:18; or
the VH includes a sequence shown in SEQ ID NO: 14, the VL includes a sequence shown in SEQ ID NO:10, and the VHH includes a sequence shown in SEQ ID NO:18.

In any of the above embodiments, the single domain antibody (VHH) in the anti-CD40×CLDN18.2 bispecific antibody of the present invention is a camelid VHH, a partially or fully humanized VHH, or a chimeric VHH.

In any of the above embodiments, the VHH of the anti-CD40×CLDN18.2 bispecific antibody of the present invention is connected to Fc through the linker. The linker may be any universal flexible sequence known in the art or obtained in the future, usually a short flexible amino acid sequence. In a specific embodiment, the linker is (G₄S) n, where n is an integer equal to or greater than 1. For example, n is an integer of 1, 2, 3, 4, 5, 6, 7, 8, or 9; GGGSG; GGSGG; GSGGG; SGGGG; GGTGS; GTSPGG; GNGGGS; G₄S-GGSGG-G₄S-SGGGG; GGG; DGGGS; TGEKP; GGRR; EGKSSGSGSESKVD; KESGSVSSEQLAQFRSLD; GGRRGGGS; LRQRDGERP; LRQKDGGGSERP; GSTSGSGKPGSGEGSTKG, etc. In a specific embodiment, the linker is selected from (G₄S)₃.

In any of the above embodiments, an Fc region of the anti-CD40×CLDN18.2 bispecific antibody of the present invention may be selected from a native Fc region of any IgG class antibody known in the prior art, such as a common or germline IgG Fc region. In a specific embodiment, the IgG Fc region may be Fc regions from different IgG subclasses, such as Fc regions from IgG1, IgG2, IgG3, and IgG4. In a specific embodiment, the IgG Fc region is an Fc sequence of IgG1. In another embodiment, the IgG Fc region may include mutation/modification to implement the function of antibody stabilization or to reduce a FcγRIIb-dependent cross-linking effect to prevent hepatotoxicity, such as the modifications known in the prior art. In a specific embodiment, the bispecific anti-CD40×CLDN18.2 antibody of the present invention is modified to include a constant region without a cross-linking effect. For example, through mutating a glycosylation site N297 residue in the Fc region to Gly, Ala, Gln, Asp, Glu, etc., the FcγRIIb-dependent cross-linking effect of the anti-CD40×CLDN18.2 bispecific antibody of the present invention can be reduced or eliminated. In a preferred embodiment, the Fc region of the anti-CD40×CLDN18.2 bispecific antibody of the present invention includes an N297Q mutation to reduce binding to an Fc receptor.

In an embodiment, a light chain constant domain CL of the anti-CD40×CLDN18.2 bispecific antibody of the present invention is from κ or λ.

In an embodiment, the present invention provides an anti-CD40×CLDN18.2 bispecific antibody, which includes:
(i) a heavy chain with a structure VH-CH1-Fc-VHH, and
(ii) a light chain with a structure VL-CL,
wherein
the heavy chain includes 3 heavy chain CDRs and CDRs contained in 3 VHHs contained in SEQ ID NO:19 and a sequence having at least 90%, 91%, 92%, 93%, 94%, 95%, 96%, 97%, 98%, 99%, or more sequence identity with SEQ ID NO:19, and the light chain includes 3 light chain CDRs contained in SEQ ID NO:23 and a sequence having at least 90%, 91%, 92%, 93%, 94%, 95%, 96%, 97%, 98%, 99%, or more sequence identity with SEQ ID NO:23;
the heavy chain includes 3 heavy chain CDRs and CDRs contained in 3 VHHs contained in SEQ ID NO:20 and a sequence having at least 90%, 91%, 92%, 93%, 94%, 95%, 96%, 97%, 98%, 99%, or more sequence identity with SEQ ID NO:20, and the light chain includes 3 light chain CDRs contained in SEQ ID NO:23 and a sequence having at least 90%, 91%, 92%, 93%, 94%, 95%, 96%, 97%, 98%, 99%, or more sequence identity with SEQ ID NO:23;
the heavy chain includes 3 heavy chain CDRs and CDRs contained in 3 VHHs contained in SEQ ID NO:21 and a sequence having at least 90%, 91%, 92%, 93%, 94%, 95%, 96%, 97%, 98%, 99%, or more sequence identity with SEQ ID NO:21, and the light chain includes 3 light chain CDRs contained in SEQ ID NO:23 and a sequence having at least 90%, 91%, 92%, 93%, 94%, 95%, 96%, 97%, 98%, 99%, or more sequence identity with SEQ ID NO:23; or
the heavy chain includes 3 heavy chain CDRs and CDRs contained in 3 VHHs contained in SEQ ID NO:22 and a sequence having at least 90%, 91%, 92%, 93%, 94%, 95%, 96%, 97%, 98%, 99%, or more sequence identity with SEQ ID NO:22, and the light chain includes 3 light chain CDRs contained in SEQ ID NO:23 and a sequence having at least 90%, 91%, 92%, 93%, 94%, 95%, 96%, 97%, 98%, 99%, or more sequence identity with SEQ ID NO:23.

In an embodiment, the present invention provides an anti-CD40×CLDN18.2 bispecific antibody, which includes:
(i) a heavy chain with a structure VH-CH1-Fc-VHH, and
(ii) a light chain with a structure VL-CL,
wherein
the heavy chain includes a sequence shown in SEQ ID NO:19, and the light chain includes a sequence shown in SEQ ID NO:23;
the heavy chain includes a sequence shown in SEQ ID NO:20, and the light chain includes a sequence shown in SEQ ID NO:23;
the heavy chain includes a sequence shown in SEQ ID NO:21, and the light chain includes a sequence shown in SEQ ID NO:23; or
the heavy chain includes a sequence shown in SEQ ID NO:22, and the light chain includes a sequence shown in SEQ ID NO:23.

In an embodiment, the anti-CD40×CLDN18.2 bispecific antibody of the present invention can enhance an immune response to the antigen and induce antibody-dependent cytotoxicity against cells expressing CD40 (for example, tumor cells expressing CD40) and cells expressing CLDN18.2 (for example, tumor cells expressing CLDN18.2).

In a second aspect, the present invention further provides a polynucleotide (nucleic acid) encoding the anti-CD40×CLDN18.2 bispecific antibody of the present invention, and a vector including the polynucleotide, preferably an expression vector.

In a third aspect, the present invention provides a host cell including the polynucleotide or the vector of the present invention. The host cell may be a prokaryotic cell or a eukaryotic cell commonly used in the art.

In a fourth aspect, the present invention provides a method for producing the anti-CD40×CLDN18.2 bispecific antibody of the present invention, including steps of (i) culturing the host cell of the present invention under conditions suitable for expressing the anti-CD40×CLDN18.2 bispecific antibody of the present invention, and optionally, (ii) recovering the anti-CD40×CLDN18.2 bispecific antibody of the present invention.

In a fifth aspect, the present invention provides an immunoconjugate, a kit, a pharmaceutical composition, a combination product, or a preparation including the anti-CD40×CLDN18.2 bispecific antibody of the present invention. In an embodiment, the pharmaceutical composition, the combination product, or the preparation provided by the present invention further includes other therapeutic agents and optional pharmaceutical aids; preferably, the other therapeutic agents are selected from chemotherapeutic agents and cytotoxic agents.

In a sixth aspect, the present invention provides a use of the anti-CD40×CLDN18.2 bispecific antibody, the immunoconjugate, the kit, the pharmaceutical composition, the combination product, or the preparation of the present invention for treating, preventing, and/or diagnosing a disease related to CD40 and a disease related to CLDN18.2. In an embodiment, the disease related to CD40 is, for example, a cancer in which CD40 is aberrantly expressed. In an embodiment, the disease related to CLDN18.2 is, for example, a cancer in which CLDN18.2 is aberrantly expressed.

In an embodiment, the present invention provides a use of the anti-CD40×CLDN18.2 bispecific antibody, the polynucleotide, the vector, the host cell, the immunoconjugate, the kit, the pharmaceutical composition, the combination product, or the preparation according to the first to third aspects and the fifth aspect in drug preparation for treating, preventing, and/or diagnosing a disease related to CD40 and a disease related to CLDN18.2.

In a seventh aspect, the present invention provides a method for treating a disease related to CD40 and a method for treating a disease related to CLDN18.2, including administering a therapeutically effective amount of the anti-CD40×CLDN18.2 bispecific antibody of the present invention or the immunoconjugate, the pharmaceutical composition, the combination product, or the preparation of the present invention to a patient in need. In an embodiment, the disease related to CD40 is, for example, a cancer in which CD40 is aberrantly expressed. In an embodiment, the disease related to CLDN18.2 is, for example, a cancer in which CLDN18.2 is aberrantly expressed.

### DESCRIPTION OF THE DRAWINGS

FIG. 1 shows a schematic structure of a bispecific antibody of the present invention.

FIG. 2 shows a binding activity of the bispecific antibody of the present invention to a P17-His fusion protein.

FIG. 3 shows a binding activity of the bispecific antibody of the present invention to huCD40-CHO-K cells.

FIG. 4 shows a binding activity of the bispecific antibody of the present invention to 18.2-HEK293 cells.

FIG. 5A and FIG. 5B show activation of CD40 signaling pathways by the bispecific antibody of the present invention in the presence of a cross-linking agent (5A) or in the absence of a cross-linking agent (5B).

FIG. 6A and FIG. 6B show activation of CD40 signaling pathways by the bispecific antibody of the present invention when CLDN18.2 is expressed (6A) or CLDN18.2 is not expressed (6B) on cell surfaces.

FIG. 7 shows an inhibitory effect of the bispecific antibody of the present invention on tumor growth in humanized mice.

FIG. 8A to FIG. 8D show results of whether the bispecific antibody of the present invention causes hepatotoxicity in humanized mice.

### DESCRIPTION OF THE EMBODIMENTS

### I. Definition

Unless defined otherwise, all technical and scientific terms used herein have the same meaning as commonly understood by one of ordinary skill in the art to which the present invention belongs. All publications, patent applications, patents, and other references mentioned herein are incorporated by reference in their entirety. In addition, materials, methods, and examples described herein are illustrative only and not intended to be limiting. Other features, objectives, and advantages of the present invention will be apparent from the description and drawings and from the appended claims.

To interpret the specification, the following definitions will apply and, wherever appropriate, terms used in the singular may also include the plural, and vice versa. It is to be understood that the terminology used herein is for the purpose of describing particular embodiments only and is not intended to be limiting.

The term “about” when used in conjunction with a numerical value is meant to encompass numerical values within a range having a lower limit that is 10% less than the specified numerical value and an upper limit that is 10% greater than the specified numerical value.

As used herein, the term “and/or” means any one of the alternatives or two or more of the alternatives.

As used herein, the terms “including” or “comprising” are meant to include stated elements, integers, or steps, but not to exclude any other elements, integers, or steps. Herein, when the term “including” or “comprising” is used, unless otherwise specified, combinations of the stated elements, integers, or steps are also encompassed. For example, when reference is made to an antibody variable region “including” a particular sequence, it is intended to also encompass an antibody variable region composed of the particular sequence.

The term “antibody” is used herein in the broadest sense to refer to a protein that includes an antigen binding site, encompassing native antibodies and artificial antibodies of various structures, including but not limited to monoclonal antibodies, polyclonal antibodies, multispecific antibodies (for example, bispecific antibodies), single chain antibodies, single domain antibodies, intact antibodies, and antibody fragments. Preferably, the antibody of the present invention is the single domain antibody or a heavy chain antibody.

The term “antibody fragment” refers to a molecule other than the intact antibody, includes a part of the intact antibody, and can bind to an antigen to which the intact antibody binds. Examples of the antibody fragment include, but are not limited to, Fv, Fab, Fab′, Fab′-SH, F(ab′)₂; double antibodies; linear antibodies; single chain antibodies (for example, scFv); single domain antibodies; bivalent or bispecific antibodies or fragments thereof; camelid antibodies (heavy chain antibodies); and bispecific antibodies or multispecific antibodies formed from antibody fragments.

The term “Fc region” includes at least part of a constant region. The term includes a native sequence Fc region and a variant Fc region. In certain embodiments, a human IgG heavy chain Fc region extends from Cys226 or Pro230 to a heavy chain carboxyl-terminus. However, C-terminal lysine (Lys447) of the Fc region may or may not be present. Unless otherwise specified, the numbering of amino acid residues in the Fc region or the constant region is according to an EU numbering system, also referred to as an EU index.

The term “variable region” or “variable domain” refers to a domain of an antibody heavy or light chain that is involved in binding the antibody to the antigen. The variable domains of the heavy and light chains of the native antibodies generally have similar structures, wherein each domain includes four conserved framework regions (FR) and 3 complementarity determining regions (CDR) (see, for example, Kindt et al. Kuby Immunology, 6th ed., WH Freeman and Co., page 91 (2007)). A single VH or VL domain may be sufficient to confer antigen binding specificity.

The “complementarity determining regions”, the “CDR regions”, or the “CDRs” are regions in an antibody variable domain that are hypervariable in sequence and form structurally defined loops (“hypervariable loops”) and/or contain antigen contacting residues (“antigen contact points”). The CDRs are mainly responsible for binding to antigenic epitopes. The heavy chain CDRs are generally referred to as CDR1, CDR2, and CDR3 and are sequentially numbered starting from the N-terminus. In a given heavy chain variable region amino acid sequence, a precise amino acid sequence boundary of each CDR may be determined using any one or a combination of many known antibody CDR assignment systems. The assignment systems include, for example, Chothia (Chothia et al. (1989) Nature 342:877-883, Al-Lazikani et al., “Standard conformations for the canonical structures of immunoglobulins”, Journal of Molecular Biology, 273, 927-948 (1997)), Kabat based on antibody sequence variability (Kabat et al., Sequences of Proteins of Immunological Interest, 4th Edition, US Department of Health and Human Services, National Institutes of Health (1987)), AbM (University of Bath), Contact (University College London), the international ImMunoGeneTics database (IMGT) (http://imgt.cines.fr/), and the North CDR definition based on affinity propagation clustering using a large number of crystal structures.

Unless otherwise specified, in the present invention, the term “CDR” or “CDR sequence” encompasses a CDR sequence determined according to any of the above manners.

The CDR may also be determined based on having the same AbM numbered position as a reference CDR sequence (for example, any of the CDRs exemplified in the present invention). In an embodiment, the position of the CDR of the antibody of the present invention is determined according to an AbM numbering scheme.

Unless otherwise specified, in the present invention, residue positions (including heavy chain variable region residues) in the antibody variable region and the CDR refer to the numbered positions according to an AbM numbering system.

The term “single domain antibody” generally refers to an antibody in which a single variable domain (for example, a heavy chain variable domain (VH) or a light chain variable domain (VL), a heavy chain variable domain derived from a camelid heavy chain antibody, or a VH-like single domain derived from fish IgNAR (v-NAR)) may confer antigen binding. That is, the single variable domain does not need to interact with another variable domain in order to recognize a target antigen. Examples of the single domain antibody include single domain antibodies derived from Camelidae (llamas and camels) and cartilaginous fish (for example, nurse sharks) (WO 2005/035572). The single domain antibody derived from Camelidae is also referred to as a VHH in the present application, is composed of only one heavy chain variable region, is an antibody that includes only one chain FR4-CDR3-FR3-CDR2-FR2-CDR1-FR1 from the C-terminus to the N-terminus, and is also referred to as a “nanobody”. The single domain antibody is the smallest unit currently known that may bind to the target antigen.

The term “multispecific antibody” refers to an antibody that has at least two antigen binding sites, and each of the at least two antigen binding sites binds to a different epitope of the same antigen or to different epitopes of different antigens. The multispecific antibody is an antibody that has binding specificities for at least two different antigenic epitopes. In an embodiment, provided herein is such a multispecific antibody, which has binding specificities for a first antigen and a second antigen, and is also referred to as the “bispecific antibody”.

The term “immunoconjugate” is an antibody conjugated to one or more other substances (including but not limited to a cytotoxic agent or a label).

The term “agonistic” refers to enabling certain parameters (for example, activity) of a given molecule (for example, a co-stimulatory molecule) to increase. For example, the term includes a substance that enables the activity of a given molecule (for example, CD40) to increase by at least 5%, 10%, 20%, 30%, 40%, or more. Therefore, agonism does not have to be 100%.

In the context involving an “anti-CD40 antibody”, the term “cross-linking effect” refers to the phenomenon that the anti-CD40 antibody binds to FcγRIIB through Fc, promoting local aggregation (multimerization) of more than one anti-CD40 antibodies, which in turn promotes aggregation of sufficient CD40 molecules. The cross-linking effect triggers transduction of downstream intracellular signals, thereby activating corresponding CD40-expressing immune cells.

The term “median effective concentration (EC₅₀)” refers to the concentration of a drug, an antibody, or a toxic agent that induces a response that is 50% between a baseline and a maximum value after a specified exposure time.

The term “therapeutic index” (TI) usually refers to a ratio of a median lethal dose (LD₅₀) to the median effective dose (ED₅₀) and is an indicator of drug safety.

Calculation of sequence identity between sequences is performed as follows.

To determine a percent identity of two amino acid sequences or two nucleic acid sequences, the sequences are compared for optimal alignment purposes (for example, gaps may be introduced in one or both of the first and second amino acids sequences or nucleic acid sequences for optimal alignment or non-homologous sequences may be discarded for alignment purposes). In a preferred embodiment, for alignment purposes, the length of the reference sequence for alignment is at least 30%, preferably at least 40%, more preferably at least 50% or 60%, and even more preferably at least 70%, 80%, 90%, or 100% of the length of the reference sequence. Amino acid residues or nucleotides at corresponding amino acid positions or nucleotide positions are then aligned. When a position in the first sequence is occupied by the same amino acid residue or nucleotide at the corresponding position in the second sequence, the molecules are identical at the position.

The sequence alignment and the calculation of percent identity between two sequences may be implemented using a mathematical algorithm. In a preferred embodiment, the percent identity between two amino acid sequences is determined using the Needlema and Wunsch ((1970) J. Mol. Biol. 48:444-453) algorithm integrated into the GAP program of the GCG software package (available at http://www.gcg.com) and using the Blossum 62 matrix or the PAM250 matrix and a gap weight of 16, 14, 12, 10, 8, 6, or 4 and a length weight of 1, 2, 3, 4, 5, or 6. In another preferred embodiment, the percent identity between two nucleotide sequences is determined using the GAP program in the GCG software package (available at http://www.gcg.com) and using the NWSgapdna. CMP matrix and a gap weight of 40, 50, 60, 70, or 80 and a length weight of 1, 2, 3, 4, 5, or 6. A particularly preferred parameter set (and a parameter set that should be used unless otherwise specified) adopts the Blossum 62 scoring matrix with a gap penalty of 12, a gap extension penalty of 4, and a frameshift gap penalty of 5.

The percent identity between two amino acid sequences or nucleotide sequences may also be determined using the PAM120 weighted remainder table, a gap length penalty of 12, and a gap penalty of 4 and using the E. Meyers and W. Miller algorithm ((1989) CABIOS, 4:11-17) incorporated into the ALIGN program (version 2.0).

Additionally or alternatively, the nucleic acid sequence and the protein sequence described herein may be further used as a “query sequence” to search against public a database to, for example, identify other family member sequences or related sequences.

The term “treatment” means to slow, interrupt, arrest, alleviate, stop, reduce, or reverse the progression or the severity of an existing symptom, disorder, condition, or disease. Desired therapeutic effects include, but are not limited to, preventing disease appearance or recurrence, alleviating symptoms, reducing any direct or indirect pathological consequences of the disease, preventing metastasis, reducing the rate of disease progression, ameliorating or palliating the disease state, and alleviating or improving prognosis. In some embodiments, the antibody of the present invention is used to delay disease development or to slow disease progression.

The term “prevention” includes the inhibition of the onset or the development of a disease or a condition or symptoms of a particular disease or condition. In some embodiments, a subject with a family history of cancer is a candidate for a preventive regimen. Generally, in the context of cancer, the term “prevention” refers to the administration of a drug before signs or symptoms of cancer occur, particularly in a subject at risk for cancer.

The term “effective amount” refers to an amount or dose of an antibody, a conjugate, or a composition of the present invention which produces a desired effect in a patient in need of treatment or prevention after administration in single or multiple doses to the patient. The effective amount may be readily determined by an attending physician, who is skilled in the art, through considering various factors, such as the species of mammal; weight, age, and general health condition; the specific illness involved; the extent or the severity of the illness; individual patient responses; the specific antibody administered; mode of administration; bioavailability characteristics of administered formulation; the dosing regimen chosen; and use of any concomitant therapy.

The term “therapeutically effective amount” refers to an amount effective to implement the desired therapeutic result at doses required and for periods of time required. The therapeutically effective amount of an antibody or an antibody fragment or a conjugate or a composition thereof may vary according to multiple factors such as the disease state, age, sex, and weight of the individual, and the ability of the antibody or a part of the antibody to elicit a desired response in the individual. The therapeutically effective amount is also an amount in which any toxic or detrimental effects of an antibody or an antibody fragment or a conjugate or a composition thereof are outweighed by therapeutically beneficial effects. Compared to an untreated subject, the “therapeutically effective amount” preferably inhibits a measurable parameter (for example, a tumor growth rate, a tumor volume, etc.) by at least about 20%, more preferably at least about 40%, even more preferably at least about 50%, 60%, or 70%, and still more preferably at least about 80% or 90%. The ability of a compound to inhibit a measurable parameter (for example, cancer) may be evaluated in an animal model system that is predictive of efficacy in human tumors.

The term “prophylactically effective amount” refers to an amount effective to implement the desired prophylactic result at doses required and for periods of time required. Typically, since a prophylactic dose is used in a subject prior to or at an earlier stage of disease, the prophylactically effective amount is less than the therapeutically effective amount.

The term “pharmaceutical composition” refers to a composition present in a form effective to permit a biological activity of an active ingredient contained therein and not including additional ingredients that are unacceptably toxic to a subject to which the composition is administered.

Terms such as “connecting peptide”, “linker”, and “peptide linker” may be used interchangeably in the present application and refer to a peptide including one or more consecutive amino acids. The amino acids are, for example, small amino acid residues or hydrophilic amino acid residues (for example, glycine, serine, threonine, proline, aspartic acid, asparagine, etc.). The connecting peptide typically includes 5 to 50 amino acids in length, such as 10, 15, 20, 25, or 30 amino acids in length. Persons skilled in the art can understand that many commonly used linkers may be used in the embodiments of the present invention.

The term “a disease related to CLDN18.2” refers to any condition caused by, aggravated, by, or otherwise related to aberrant expression (for example, increased expression) or aberrant activity of CLDN18.2 (for example, human CLDN18.2). In an embodiment, the disease related to CLDN18.2 is a tumor in which CLDN18.2 (for example, human CLDN18.2) is aberrantly expressed (for example, increasingly expressed).

The term “a disease related to CD40” refers to any condition caused by, aggravated by, or otherwise related to aberrant expression (for example, increased expression) or aberrant activity of CD40. In an embodiment, the disease related to CD40 is a tumor in which CD40 is aberrantly expressed (for example, increasingly expressed).

The terms “individual” and “subject” may be used interchangeably and include mammals. The mammals include, but are not limited to, domesticated animals (for example, cows, sheep, cats, dogs, and horses), primates (for example, humans and non-human primates such as monkeys), rabbits, and rodents (for example, mice and rats). In particular, the individual or the subject is a human.

### II. Antibody of the Present Invention

Claudin 18.2 (also referred to as “CLDN18.2” herein) has significant differences in expression between cancer tissues and normal tissues, which may be due to the fact that a CREB binding site in a promoter region of Claudin 18.2 is highly methylated by CpG in normal tissues, while the CpG methylation level decreases during cell carcinogenesis, and CREB is involved in activating the transcription of Claudin18.2.

The “bispecific antibody against CD40 and CLDN18.2”, “bispecific antibody specifically binding to CD40 and CLDN18.2”, “anti-CD40×CLDN18.2 bispecific antibody”, “CD40/CLDN18.2 bispecific antibody”, and similar terms mentioned herein refer to the bispecific antibody that can bind to the targets CD40 and Claudin 18.2 with sufficient affinity. The bispecific antibody can recruit immune cells and redirect the lysis of target cells. The bispecific antibody can implement high-intensity activation of immune cells specifically at a lesion site, while achieving low agonistic activity in locations such as hepatocytes that cannot form cross-linking effects, so as to implement specific killing of tumor cells without causing adverse events such as hepatotoxicity.

In an embodiment, the bispecific anti-CD40×CLDN18.2 antibody of the present invention includes an amino acid modification, such as an amino acid substitution, addition, or deletion, preferably the amino acid substitution, and more preferably an amino acid conservative substitution.

In an embodiment, the amino acid modification described in the present invention occurs in a region (for example, in FR) outside CDR. In an embodiment, the substitution is the conservative substitution. The conservative substitution refers to substitution of one amino acid with another amino acid within the same class, such as substitution of one acidic amino acid with another acidic amino acid, one basic amino acid with another basic amino acid, or one neutral amino acid with another neutral amino acid.

Exemplary substitutions are shown in the following table:

| Original | | Preferred conservative |
|---|---|---|
| residue | Exemplary substitution | amino acid substitution |
| Ala (A) | Val, Leu, Ile | Val |
| Arg (R) | Lys, Gln, Asn | Lys |
| Asn (N) | Gln, His, Asp, Lys, Arg | Gln |
| Asp (D) | Glu, Asn | Glu |
| Cys (C) | Ser, Ala | Ser |
| Gln (Q) | Asn, Glu | Asn |
| Glu (E) | Asp, Gln | Asp |
| Gly (G) | Ala | Ala |
| His (H) | Asn, Gln, Lys, Arg | Arg |
| Ile (I) | Leu, Val, Met, Ala, Phe, | Leu |
| | Norleucine | |
| Leu (L) | Norleucine, Ile, Val, Met, | Ile |
| | Ala, Phe | |
| Lys (K) | Arg, Gln, Asn | Arg |
| Met (M) | Leu, Phe, Ile | Leu |
| Phe (F) | Trp, Leu, Val, Ile, Ala, Tyr | Tyr |
| Pro (P) | Ala | Ala |
| Ser (S) | Thr | Thr |
| Thr (T) | Val, Ser | Ser |
| Trp (W) | Tyr, Phe | Tyr |
| Tyr (Y) | Trp, Phe, Thr, Ser | Phe |
| Val (V) | Ile, Leu, Met, Phe, Ala, | Leu |
| | Norleucine | |

In certain embodiments, the substitution occurs in the CDR region of the antibody. Typically, a variant obtained has a modification (for example, improvement) in certain biological properties (for example, increased affinity) relative to a parent antibody and/or has substantially retained certain biological properties of the parent antibody. An exemplary substitutional variant is an affinity matured antibody.

In certain embodiments, one or more amino acid modifications may be introduced into the Fc region of the antibody provided herein to produce an Fc region variant. The Fc region variant may include a human Fc region sequence (for example, a human IgG1, IgG2, IgG3, or IgG4 Fc region) including the amino acid modification (for example, the substitution) at one or more amino acid positions.

Studies have found that the Fc regions of some agonistic anti-CD40 antibodies form cross-linking effects with FcγRIIB abundantly expressed on surfaces of hepatocytes, causing non-specific activation of immune cells expressing CD40 on surfaces of hepatocytes, thereby causing hepatotoxicity. Therefore, the anti-CD40×CLDN18.2 bispecific antibody of the present invention may also include the modification in the Fc region that reduces the binding affinity of the antibody of the present invention to FcγRIIb, so as to reduce or eliminate the cross-linking effect caused by the Fc region. In an embodiment, the modification is in a CH2 domain of the Fc region, such as at position 329 (EU numbering) of a heavy chain (for example, P329G). In an embodiment, the bispecific anti-CD40×CLDN18.2 antibody of the present invention includes amino acid substitutions at positions 234 and 235 (EU numbering) of the heavy chain. In a specific embodiment, the amino acid substitutions are L234A and L235A (also referred to as “LALA mutations”).

In an embodiment, the anti-CD40×CLDN18.2 bispecific antibody of the present invention can enhance an immune response independent of the binding of the antibody to an Fc receptor. For example, the bispecific anti-CD40×CLDN18.2 antibody of the present invention can exhibit potent CD40 agonistic characteristics without cross-linking with the Fc receptor, such as FcγR.

In an embodiment, a disulfide bond is present between CH1 and CL of the anti-CD40×CLDN18.2 bispecific antibody of the present invention. In an embodiment, the number of disulfide bonds may vary depending on the IgG form from which the constant domain of the antibody is derived. In some embodiments, there are 2 or 4 disulfide bonds between hinge regions.

In certain embodiments, a cysteine engineered antibody, such as “thio mAb”, may need to be produced, wherein one or more residues of the antibody are substituted by cysteine residues.

### III. Nucleic Acid and Vector and Host Cell Including the Same of the Present Invention

In an aspect, the present invention provides a nucleic acid encoding the bispecific antibody according to any of the above or an antigen binding fragment thereof. The present invention also encompasses a nucleic acid that hybridizes to the nucleic acid under stringent conditions, a nucleic acid that has one or more substitutions (for example, conservative substitutions), deletions, or insertions compared to the nucleic acid, or a nucleic acid sequence at least 80%, at least 85%, or at least 90%, 91%, 92%, 93%, 94%, 95%, 96%, 97%, 98%, 99%, or more identical to the nucleic acid.

In another aspect, the present invention provides a vector including the nucleic acid. In a preferred embodiment, the vector is an expression vector. Persons skilled in the art can fully understand that vector commonly used in the art to which the present invention belongs may be applied to the present invention.

In an embodiment, the present invention provides a host cell including the nucleic acid or the vector.

The term “host cell” refers to a cell into which an exogenous polynucleotide is introduced, including progeny of such a cell. The host cell includes a “transformant” and a “transformed cells”, which include a primary transformed cell and progeny derived therefrom without regard to the number of passages. The progeny may not be completely identical in nucleic acid content to a parent cell and may include mutations. Mutant progeny screened or selected for the same function or biological activity in the originally transformed cell is included herein. The host cells are any type of cell system that may be used to produce antibody molecules of the present invention and include eukaryotic cells, such as mammalian cells (for example, CHO cells or HEK293 cells), insect cells, yeast cells; and prokaryotic cells, such as E. coli cells. The host cells include cultured cells and may also include cells within transgenic animals, transgenic plants, or cultured plant tissues or animal tissues.

### IV. Composition of the Present Invention

In some embodiments, the present invention provides a composition including the anti-CD40×CLDN18.2 bispecific antibody or the antigen binding fragment thereof according to any of the above, preferably, the composition is a pharmaceutical composition. In an embodiment, the composition further includes a pharmaceutical aid. The term “pharmaceutical aid” refers to a diluent, an adjuvant, a vector, an excipient, a stabilizer, etc. administered together with an active substance.

In an embodiment, the composition (for example, the pharmaceutical composition) includes the anti-CD40×CLDN18.2 bispecific antibody or the antigen binding fragment thereof of the present invention and a combination of one or more other therapeutic agents (for example, chemotherapeutic agents, cytotoxic agents, vaccines, other antibodies, anti-infective agents, small molecule drugs, or immunomodulators).

The pharmaceutical composition of the present invention may also include one or more other therapeutic agents. The therapeutic agents are required for particular indications being treated, encompassing any substance effective in preventing or treating tumors (for example, cancer) and infections (for example, chronic infections), preferably the therapeutic agents that do not adversely affect activities of one another. For example, ideally, other anti-cancer active therapeutic agents, such as chemotherapeutic agents, cytotoxic agents, vaccines, other antibodies, anti-infective active agents, small molecule drugs, and immunomodulatory agents, are also provided. The therapeutic agents are suitably present in combination in amounts that are effective for an intended application.

### V. Preparation of Anti-CD40×CLDN18.2 Bispecific Antibody of the Present Invention

In an embodiment, the present invention provides a method for preparing an anti-CD40×CLDN18.2 bispecific antibody, wherein the method includes culturing a host cell including a nucleic acid encoding the anti-CD40×CLDN18.2 bispecific antibody or an expression vector including the nucleic acid under conditions suitable for expressing the nucleic acid encoding the anti-CD40×CLDN18.2 bispecific antibody, and optionally isolating the anti-CD40×CLDN18.2 bispecific antibody. In a certain embodiment, the method further includes recovering the anti-CD40×CLDN18.2 bispecific antibody from the host cell (or a host cell culture medium).

In order to recombinantly produce the anti-CD40×CLDN18.2 bispecific antibody of the present invention, the nucleic acid encoding the anti-CD40×CLDN18.2 bispecific antibody of the present invention is first isolated and the nucleic acid is inserted into the vector for further cloning and/or expression in the host cell. Such a nucleic acid is readily isolated and sequenced using a conventional procedure, such as through using an oligonucleotide probe that can specifically bind to the nucleic acid encoding the anti-CD40×CLDN18.2 bispecific antibody of the present invention.

The anti-CD40×CLDN18.2 bispecific antibody of the present invention prepared as described herein may be purified through known prior art techniques such as high performance liquid chromatography, ion exchange chromatography, gel electrophoresis, affinity chromatography, and size exclusion chromatography. Actual conditions used to purify a particular protein also depend on factors such as net charge, hydrophobicity, and hydrophilicity, and will be apparent to persons skilled in the art. The purity of the anti-CD40×CLDN18.2 bispecific antibody of the present invention may be determined through any of various known analytical methods. The known analytical method includes size exclusion chromatography, gel electrophoresis, high performance liquid chromatography, etc.

### VI. Combination Product or Kit

In some embodiments, the present invention also provides a combination product, which includes the anti-CD40×CLDN18.2 bispecific antibody or the antigen binding fragment of the present invention or an immunoconjugate thereof and one or more other therapeutic agents (for example, chemotherapeutic agents, other antibodies, cytotoxic agents, anti-infective agents, small molecule drugs, immunomodulators, etc.).

In some embodiments, the combination product is used to prevent or treat a tumor. In some embodiments, the tumor is cancer, etc.

In some embodiments, two or more ingredients of the combination product may be sequentially, separately, or simultaneously co-administered to a subject.

In some embodiments, the present invention also provides a kit including the anti-CD40×CLDN18.2 bispecific antibody, the pharmaceutical composition, the immunoconjugate, or the combination product of the present invention, and optionally a package insert for guiding administration.

In some embodiments, the present invention also provides a pharmaceutical preparation including the anti-CD40×CLDN18.2 bispecific antibody, the pharmaceutical composition, the immunoconjugate, or the combination product of the present invention, optionally, the pharmaceutical preparation further includes a package insert for guiding administration.

### VII. Use and Treatment Method of Anti-CD40×CLDN18.2 Bispecific Antibody of the Present Invention

In an aspect, the present invention relates to a method for modulating an immune response in an individual. The method includes administering an effective amount of the anti-CD40×CLDN18.2 bispecific antibody or the pharmaceutical composition, the immunoconjugate, or the combination product including the anti-CD40×CLDN18.2 bispecific antibody disclosed herein to a subject, thereby anchoring a CD40 antibody that can activate B cells to cells expressing CLDN18.2, thereby activating the B cells and downstream immune response cells such as T cells to enhance the killing effect, so as to improve the weak antibody-dependent cell-mediated cytotoxicity (ADCC) killing effect of the current CLDN18.2 monoclonal antibody. The mechanism is that the anti-CD40×CLDN18.2 bispecific antibody produces a cross-linking effect, thereby significantly producing an immune activation reaction when the cross-linking effect is formed, and producing no or only a weak immune activation effect when there is no cross-linking effect.

In an embodiment, a therapeutically effective amount of the anti-CD40×CLDN18.2 bispecific antibody or the pharmaceutical composition, the immunoconjugate, or the combination product disclosed herein restores, enhances, stimulates, or increases the immune response in the subject.

In an embodiment, when the anti-CD40×CLDN18.2 bispecific antibody disclosed herein binds to CLDN18.2 on a cell surface, the agonistic activity is significantly higher than the agonistic activity without binding to the cell surface, which can reduce non-specific agonistic activity and improve the therapeutic index to the greatest extent.

In an embodiment, the anti-CD40×CLDN18.2 bispecific antibody disclosed herein does not bind to the Fc receptor on the liver after Fc fragment modification. In addition, the bispecific antibody of the present invention has an activation effect that is dependent on the CLDN18.2 target, and the CLDN18.2 target is a tumor-specific target, so the anti-CD40×CLDN18.2 bispecific antibody of the present invention can completely prevent hepatotoxicity from being produced.

In another aspect, the present invention relates to a method for preventing or treating a tumor (for example, cancer) in a subject. The method includes administering an effective amount of the anti-CD40×CLDN18.2 bispecific antibody or the pharmaceutical composition, the immunoconjugate, or the combination product including the same disclosed herein to the subject.

In some embodiments, the tumor is a cancer in which CLDN 18.2 is aberrantly expressed. In some embodiments, the cancer in which CLDN 18.2 is aberrantly expressed is, for example, bone cancer, blood cancer, lung cancer, liver cancer, pancreatic cancer, esophageal cancer, skin cancer, head and neck cancer, skin or intraocular melanoma, uterine cancer, ovarian cancer, rectal cancer, anal cancer, stomach cancer, colon cancer, breast cancer, prostate cancer, uterine cancer, sex and reproductive organ cancer, Hodgkin's disease, esophageal cancer, small intestine cancer, endocrine system cancer, thyroid cancer, parathyroid cancer, adrenal cancer, soft tissue sarcoma, bladder cancer, kidney cancer, renal cell carcinoma, renal pelvis cancer, central nervous system (CNS) tumor, neuroectodermal cancer, spinal axis tumor, gliomas, or meningiomas and pituitary adenomas, preferably, the cancer is stomach cancer, pancreatic cancer, esophageal cancer, ovarian cancer, or lung cancer.

In some embodiments, the tumor is a cancer in which CD40 is aberrantly expressed. In some embodiments, the cancer includes, but is not limited to, solid tumor, hematological cancer, soft tissue tumor, and metastatic lesion. Examples of the solid tumor includes malignancies, such as sarcomas and carcinomas (including adenocarcinomas and squamous cell carcinomas) of multiple organ systems, such as cancers that invade liver, lung, breast, lymphoid, gastrointestinal tract (for example, colon), pancreas, genitourinary tract (for example, kidney, bladder epithelial cells), prostate, and pharynx. The adenocarcinomas include malignancies such as most colon cancers, rectal cancers, renal cell carcinomas, liver cancers, non-small cell lung cancers, small bowel cancers, and esophageal cancers. The squamous cell carcinomas include malignancies such as cancers in lung, esophagus, skin, head and neck area, mouth, anus, and cervix. In an embodiment, the cancer is melanoma, such as advanced melanoma. In an embodiment, the cancer is lymphoma, renal cell carcinoma, non-small cell lung cancer, liver cancer, pancreatic cancer, colon adenocarcinoma, or breast cancer. The metastatic lesions of the above cancers may also be treated or prevented using the method and the composition of the present invention.

Non-limiting examples of preferred cancers for treatment include lymphoma (for example, diffuse large B-cell lymphoma, Hodgkin's lymphoma, or non-Hodgkin's lymphoma), breast cancer (for example, metastatic breast cancer), liver cancer (for example, hepatocellular carcinoma (HCC)), lung cancer (for example, non-small cell lung cancer (NSCLC), such as stage IV or recurrent non-small cell lung cancer, NSCLC adenocarcinoma, or NSCLC squamous cell carcinoma), myeloma (for example, multiple myeloma), leukemia (for example, chronic myeloid leukemia), skin cancer (for example, melanoma (for example, stage III or stage IV melanoma) or Merkel cell carcinoma), head and neck cancer (for example, head and neck squamous cell carcinoma (HNSCC)), myelodysplastic syndrome, bladder cancer (for example, transitional cell carcinoma), kidney cancer (for example, renal cell carcinoma, such as clear cell renal cell carcinoma, such as advanced or metastatic clear cell renal cell carcinoma), and colon cancer. Additionally, refractory or recurrent malignancies (for example, pancreatic cancer) may be treated using the anti-CD40 antibodies or the pharmaceutical composition, the immunoconjugate, or the combination product including the same described herein.

The subject may be a mammal, such as a primate, preferably, a higher primate, such as human (for example, a patient having or at risk of having a disease described herein). In an embodiment, the subject has or is at risk of having a disease described herein (for example, a tumor as described herein). In certain embodiments, the subject is receiving or has received other treatments, such as chemotherapy and/or radiation therapy. Alternatively or in combination, the subject is or is at risk of being immunocompromised due to an infection.

In some embodiments, the prevention or treatment method described herein further include administering the anti-CD40×CLDN18.2 bispecific antibody or the pharmaceutical composition, the immunoconjugate, or the combination product disclosed herein to the subject or the individual and one or more other therapies, such as treatment modalities and/or other therapeutic agents.

The anti-CD40×CLDN18.2 bispecific antibody of the present invention (and the pharmaceutical composition or the immunoconjugate including the same) may be administered through any suitable method, including parenteral administration, intrapulmonary administration, and intranasal administration, and if local treatment is required, intralesional administration. Parenteral infusions include intramuscular, intravenous, intraarterial, intraperitoneal, or subcutaneous administration. Administration may be through any suitable way, such as through injection, for example, intravenous or subcutaneous injection, depending in part on whether the administration is brief or chronic. Various dosing schedules are encompassed herein and include, but are not limited to, single administration or multiple administrations over multiple time points, bolus administration, and pulse infusion.

For the prevention or the treatment of a disease, the appropriate dose of the anti-CD40×CLDN18.2 bispecific antibody of the present invention (when used alone or in combination with one or more other therapeutic agents) depends on the type of the disease to be treated, the type of the anti-CD40×CLDN18.2 bispecific antibody, the severity and the course of the disease, whether the anti-CD40×CLDN18.2 bispecific antibody is administered for preventive or therapeutic purposes, previous treatments, the clinical history and the response to the anti-CD40×CLDN18.2 bispecific antibody of the patient, and the judgment of the attending physician. The anti-CD40×CLDN18.2 bispecific antibody is suitably administered to the patient as a single treatment or over a series of treatments. The dose and the treatment regimen of the anti-CD40×CLDN18.2 bispecific antibody may be determined by a skilled person.

It is to be understood that any of the above prevention or treatment may be performed using the immunoconjugate, the composition, or the combination product of the present invention instead of the anti-CD40×CLDN18.2 bispecific antibody.

The following examples are described to facilitate understanding of the present invention. The examples are not intended to, and should not be interpreted in any way as, limiting the protection scope of the present invention.

### EXAMPLE

Unless otherwise specified, the experimental methods used in the following examples are all methods of conventional chemistry, biochemistry, organic chemistry, molecular biology, microbiology, recombinant DNA technology, genetics, immunology, and cell biology within the technical scope of the art. Unless otherwise specified, materials, reagents, etc. used in the following examples may be obtained from commercial sources.

### Example 1. Preparation of Anti-CD40×CLDN18.2 Bispecific Antibody

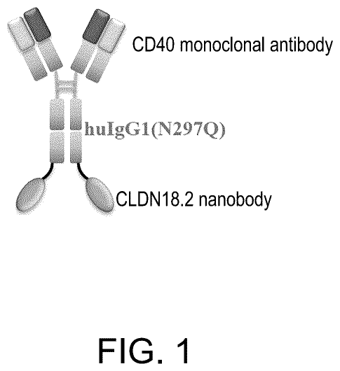

Anti-CD40 antibodies C8-WT, C8-2, C8-6, and C8-10 in Patent Application No. CN202110577432.9 were respectively constructed with VHH of an anti-CLDN18.2 antibody NA3SH1-T4-hVH6 in Patent Application No. CN202110795793.0 to form a bispecific antibody with the structure shown in FIG. 1, wherein sequences of the antibodies are shown in Tables 1a to 1d.

TABLE 1a
CDR sequence in light chain variable
region of anti-CD40 antibody (defined
by AbM numbering)

| Anti- | SEQ | | SEQ | | SEQ | |
|---|---|---|---|---|---|---|
| body | ID | | ID | | ID | |
| name | NO: | LCDR1 | NO: | LCDR2 | NO: | LCDR3 |
| C8-WT | 1 | RSSQGIRSSLA | 2 | GASSLEG | 3 | QQLNSFPST |
| C8-2 | 1 | RSSQGIRSSLA | 2 | GASSLEG | 3 | QQLNSFPST |
| C8-6 | 1 | RSSQGIRSSLA | 2 | GASSLEG | 3 | QQLNSFPST |
| C8-10 | 1 | RSSQGIRSSLA | 2 | GASSLEG | 3 | QQLNSFPST |

TABLE 1b
CDR sequence in heavy chain variable
region of anti-CD40 antibody (defined
by AbM numbering)

| Anti- | SEQ | | SEQ | | SEQ | |
|---|---|---|---|---|---|---|
| body | ID | | ID | | ID | |
| name | NO: | HCDR1 | NO: | HCDR2 | NO: | HCDR3 |
| C8-WT | 4 | GFTFGSYEMN | 5 | YISSSGETTD | 6 | DVFFFDSSG |
| | | | | | | DNGAYGMDV |
| C8-2 | 4 | GFTFGSYEMN | 5 | YISSSGETTD | 7 | DVFFFSPSG |
| | | | | | | DNGAYGMDV |
| C8-6 | 4 | GFTFGSYEMN | 5 | YISSSGETTD | 8 | DVFFFDSSG |
| | | | | | | SPGAYGMDV |
| C8-10 | 4 | GFTFGSYEMN | 5 | YISSSGETTD | 9 | DVFFFDSSG |
| | | | | | | FPGAYGMDV |

TABLE 1c
sequence of heavy chain variable region and light chain
variable region of anti-CD40 antibody

| Antibody | | |
|---|---|---|
| name | Light chain variable region | Heavy chain variable region |
| C8-WT | DIQLTQSPSSLTASVGDRVTITCRSSQGIR | EVQLLESGGGLVRPGGSLRLSCAASGFTF |
| | SSLAWYQQKPGKPPKLLIYGASSLEGGVPS | GSYEMNWVRQAPGKGLEWVSYISSSGETT |
| | RFSGSGSGTDFTLTISSLQPEDFATYYCQQ | DYADSVKGRFTVSRDNSKNLLYLQMNSLR |
| | LNSFPSTFGQGTKLEIK (SEQ ID NO: | AEDTAVYYCARDVFFFDSSGDNGAYGMDV |
| | 10) | WGQGTMVTVSS (SEQ ID NO: 11) |
| C8-2 | SEQ ID NO: 10 | EVQLLESGGGLVRPGGSLRLSCAASGFTF |
| | | GSYEMNWVRQAPGKGLEWVSYISSSGETT |
| | | DYADSVKGRFTVSRDNSKNLLYLQMNSLR |
| | | AEDTAVYYCARDVFFFSPSGDNGAYGMDV |
| | | WGQGTMVTVSS (SEQ ID NO: 12) |
| C8-6 | SEQ ID NO: 10 | EVQLLESGGGLVRPGGSLRLSCAASGFTF |
| | | GSYEMNWVRQAPGKGLEWVSYISSSGETT |
| | | DYADSVKGRFTVSRDNSKNLLYLQMNSLR |
| | | AEDTAVYYCARDVFFFDSSGSPGAYGMDV |
| | | WGQGTMVTVSS (SEQ ID NO: 13) |
| C8-10 | SEQ ID NO: 10 | EVQLLESGGGLVRPGGSLRLSCAASGFTF |
| | | GSYEMNWVRQAPGKGLEWVSYISSSGETT |
| | | DYADSVKGRFTVSRDNSKNLLYLQMNSLR |
| | | AEDTAVYYCARDVFFFDSSGFPGAYGMDV |
| | | WGQGTMVTVSS (SEQ ID NO: 14) |

TABLE 1d
sequence of anti-CLDN18.2 nanobody
CDR sequence (defined by AbM numbering)

| CDR1 | CDR2 | CDR3 |
|---|---|---|
| GSIFHIPVMG | GISRGGTTN | LVVSGIGSTLEV |
| (SEQ ID NO: 15) | (SEQ ID NO: 16) | (SEQ ID NO: 17) |

Nanobody sequence
QVQLVESGGGLVQPGGSLRLSCAASGSIFHIPVMGWYRQAPGKGLELVAG
ISRGGTTNYADSVKGRFTISRDNAKNTLYLQMNSLRAEDTAVYYCNVLVV
SGIGSTLEVWGQGTLVTVSS (SEQ ID NO: 18)

Specifically, the obtained nucleotide sequences encoding the heavy chain variable regions of the above 4 anti-CD40 antibodies and the nucleotide sequences encoding the light chain variable regions of the anti-CD40 antibodies were respectively spliced with the nucleotide sequences encoding heavy and light chain constant region fragments to obtain corresponding full-length heavy chain and light chain encoding nucleotide sequences of the anti-CD40 antibodies. In order to reduce the binding of the antibody of the present invention to the Fc receptor on the hepatocytes, the Fc of the bispecific antibody adopted an hIgG1 (N297Q) Fc isoform. Then, the nucleotide sequence encoding VHH of the anti-CLDN18.2 antibody was respectively connected to the C-terminus of the heavy chain constant regions of 4 different anti-CD40 antibodies through a linker sequence, thereby obtaining the heavy chain sequence of the bispecific antibody shown in FIG. 1, wherein the linker sequence encoded a flexible linker commonly used in the art for connecting different peptide molecules, such as the linker shown as GGGGSGGGGSGGGGS (SEQ ID NO:25). The obtained encoding nucleotide sequence was then constructed onto a eukaryotic expression vector plasmid pcDNA3.4. The heavy chain and the light chain of the bispecific antibody were co-expressed in CHO-K cells using the ExpiCHO transient expression system (Thermo Fisher, A29133) according to instructions from the manufacturer. Then, affinity purification was performed through COLUMN XK16/20 (purchased from Cytiva) to respectively obtain the anti-CD40×CLDN18.2 bispecific antibodies C8-WT-NA3S-HC, C8-2-NA3S-HC, C8-6-NA3S-HC, and C8-10-NA3S-HC with heavy and light chain amino acid sequence numbers shown in Table 2.

TABLE 2
Amino acid sequence of anti-CD40 ×
CLDN18.2 bispecific antibody

| Antibody name | Light chain | Heavy chain |
|---|---|---|
| C8-WT-NA3S-HC | SEQ ID NO: 23 | SEQ ID NO: 19 |
| C8-2-NA3S-HC | SEQ ID NO: 23 | SEQ ID NO: 20 |
| C8-6-NA3S-HC | SEQ ID NO: 23 | SEQ ID NO: 21 |
| C8-10-NA3S-HC | SEQ ID NO: 23 | SEQ ID NO: 22 |

### Example 2. Affinity Activity Analysis of Anti-CD40×CLDN18.2 Bispecific Antibody

### 2.1 Binding Ability of Bispecific Antibodies to CD40 Protein

The obtained 4 bispecific antibodies were detected for affinity against the extracellular domain of human CD40 (Uniprot: positions 21 to 193 of P25942) through ELISA. The extracellular domain of human CD40 carried a His tag, also referred to herein as a P17-His fusion protein.

2 μg/mL of the P17-His fusion protein was coated onto a 96-well ELISA plate at 30 μL per well at 4° C. overnight. The next day, after encapsulating in 5% PBSM (PBS+5% Milk) at room temperature for 2 h, serially diluted (20 nM in the first well, 10-fold dilution in the second well, 3-fold dilution in the third to seventh wells, and 4-fold dilution in the seventh to eighth wells) anti-CD40×CLDN18.2 bispecific antibody, positive control APX005 (sequence derived from Apexigen Inc. US20120301488A1, prepared in the laboratory), or isotype IgG1 negative control were added. After culturing at room temperature for 1 h, washed 3 times with PBST (PBS+0.05% Tween-20), and then 30 μL of Goat-anti-human-Fc-HRP (abcam, ab97225) was added. After culturing at room temperature for 1 h, washed 3 times with PBST (PBS+0.05% Tween-20), and TMB colorimetric solution was added for color development for 5 to 30 min. After termination, OD450 was detected through a microplate reader (Beckman Coulter), and the results were analyzed and plotted through Graphpad 7.0 to evaluate the binding of the bispecific antibody to the P17-His fusion protein.

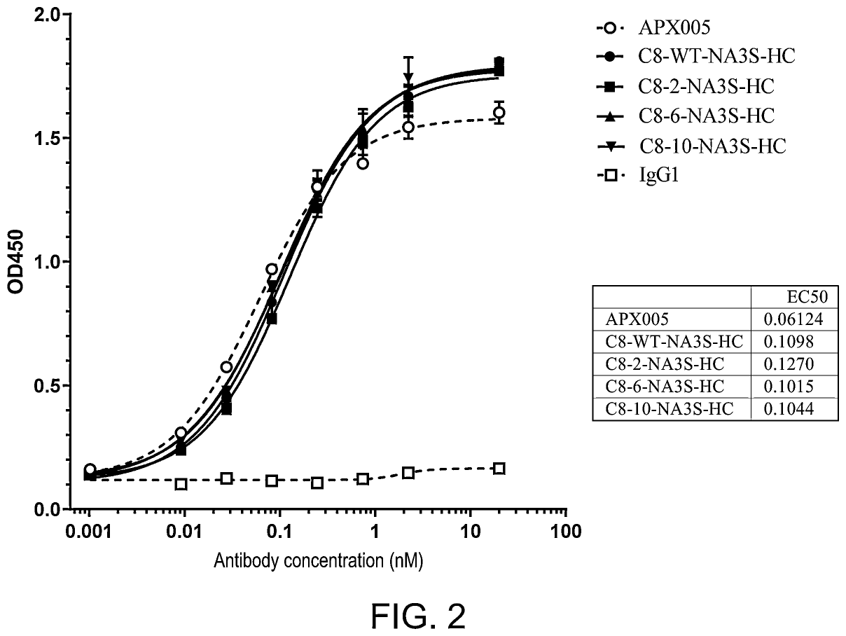

The results are shown in FIG. 2. As can be seen from FIG. 2, the EC₅₀ value of the binding of the positive control APX005 to the P17-His fusion protein is 0.06124 nM, and the EC₅₀ values of the binding of the bispecific antibodies C8-WT-NA3S-HC, C8-2-NA3S-HC, C8-6-NA3S-HC, and C8-10-NA3S-HC to the P17-His fusion protein are respectively 0.1098 nM, 0.1270 nM, 0.1015 nM, and 0.1044 nM, showing affinities comparable to that of the positive control APX005.

### 2.2 Binding Ability of Bispecific Antibody to CD40 Expressed on Cell Surface

Furthermore, in order to test the binding ability of the bispecific antibody to CD40 expressed on the cell surface, CHO-K cells (Thermo Fisher) expressing human CD40 (Uniprot: P25942) on the cell surface were prepared. Specifically, the human CD40 encoding sequence was cloned into a multiple cloning site of a pcDNA3.4 (Invitrogen) vector. Then, an expression vector expressing human CD40 was introduced into the CHO-K cells for eukaryotic expression, thereby obtaining the CHO-K cells expressing human CD40 on the cell surface (hereinafter also referred to as huCD40-CHO-K cells).

The huCD40-CHO-K cells were seeded into a 96-well plate at 1.0×10⁵ cells/well, and serially diluted (115.6 nM in the first well, 10-fold dilution in the second well, 3-fold serial dilution in the third to seventh wells, and 4-fold dilution in the seventh to eighth wells) anti-CD40×CLDN18.2 bispecific antibody was added, wherein the positive control was APX005 and the negative control was isotype IgG1. After culturing at 4° C. for 30 min, the cells were washed and then added with 100 μL of 1:300 diluted Goat F(ab′)₂ Anti-human Fc (PE) (Abcam, ab98596) and cultured for 30 min. The cells were then washed and the binding of the bispecific antibody of the present invention to CD40 expressed on the cell surface was detected through flow cytometry (Beckman Coulter).

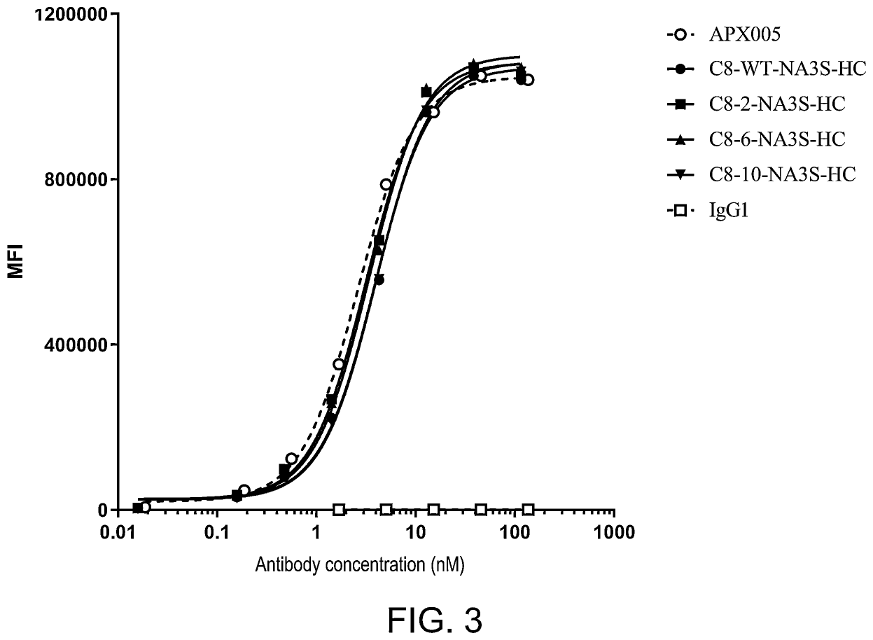

The detection results are shown in FIG. 3. FIG. 3 shows that different concentrations of the anti-CD40×CLDN18.2 bispecific antibody exhibited binding ability to the huCD40-CHO-K cells, and within a bispecific antibody concentration range of 0.015 nM to 115.6 nM, the binding ability was concentration-dependent.

### 2.3 Affinity of Bispecific Antibody to CLDN18.2 Expressed on Cell Surface

In order to test the affinity of the bispecific antibody to CLDN18.2 expressed on the cell surface, HEK293 cells expressing human CLDN18.2 on the cell surface were prepared. Specifically, a DNA sequence of full-length human CLDN18.2 (see SEQ ID NO:15 of WO2020238730A1 for amino acid sequence) was constructed into pLVX-puro plasmid (Clontech, Cat #632164). The obtained plasmid was then transformed into HEK293 cells (ATCC®CRL-1573™) through electroporation. Through performing resistance pressure screening with puromycin and identifying clones using an antibody IMAB362 (Ganymed, Germany, an antibody specifically binding to CLDN18.2), a HEK293 cell line overexpressing human CLDN18.2, also referred to as “18.2-HEK293 cells” herein, was finally obtained.

The 18.2-HEK293 cells were seeded into a 96-well plate at 1.0×10⁵ cells/well, and serially diluted (115.6 nM in the first well, 10-fold dilution in the second well, 3-fold serial dilution in the third to seventh wells, and 4-fold dilution in the seventh to eighth wells) bispecific antibody of the present invention, a positive control NA3SH1-T4-hVH6 (amino acid sequence shown in SEQ ID NO:24), or isotype IgG1 negative control was added. After culturing at 4° C. for 30 min, the cells were washed and added with 100 μL of 1:300 diluted Goat F(ab′)₂ Anti-human Fc (PE) and cultured for 30 min. The cells were then washed and the binding of the bispecific antibody to CLDN18.2 expressed on the surface of HEK293 cells was detected through flow cytometry.

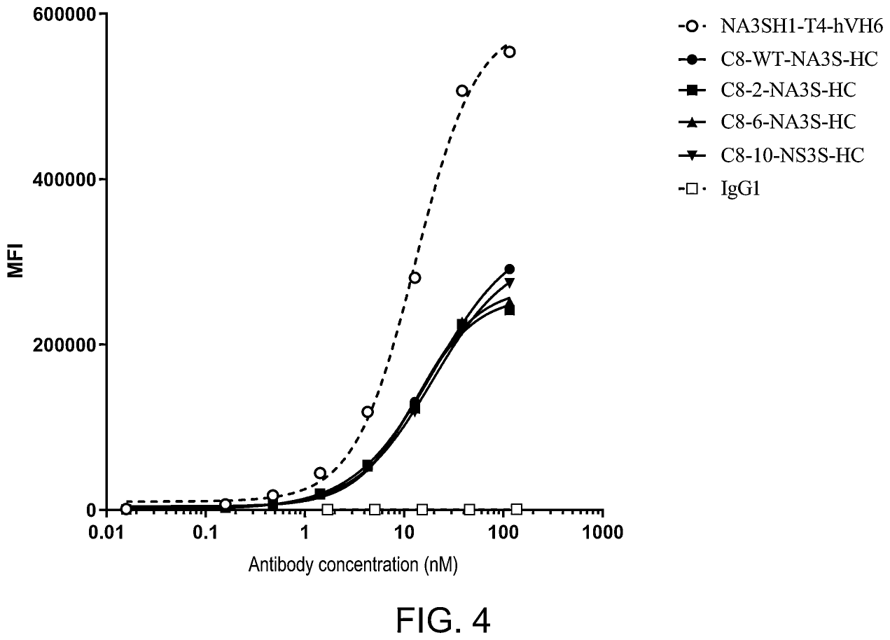

FIG. 4 shows that different concentrations of the anti-CD40×CLDN18.2 bispecific antibody bound to the 18.2-HEK293 cells, and within the bispecific antibody concentration range of 0.015 nM to 115.6 nM, the binding affinity was bispecific antibody concentration-dependent.

### 2.4 Binding Ability of Bispecific Antibody to CLDN18.1 Expressed on Cell Surface

In order to test the affinity of the bispecific antibody to CLDN18.1 expressed on the cell surface, HEK293 cells expressing human CLDN18.1 (see SEQ ID NO:16 of WO2020238730A1 for amino acid sequence) on the cell surface were prepared (the preparation method is described above, hereinafter also referred to as “18.1-HEK293 cells”).

The 18.1-HEK293 cells were seeded into a 96-well plate at 1.0×10⁵ cells/well, and serially diluted (115.6 nM in the first well, 10-fold dilution in the second well, 3-fold serial dilution in the third to seventh wells, and 4-fold dilution in the seventh to eighth wells) bispecific antibody of the present invention or a positive antibody NA3SH1-T4-hVH6 was added. After culturing at 4° C. for 30 min, the cells were washed and added with 100 μL of 1:300 diluted PE-labeled Goat F(ab′)₂ Anti-human Fc (PE) and cultured at 4° C. for 30 min. The cells were then washed and the binding of the bispecific antibody to CLDN18.1 molecules expressed on the surface of HEK293 cells was detected through flow cytometry.

The results showed that different concentrations of the anti-CD40×CLDN18.2 bispecific antibody did not bind to the 18.1-HEK293 cells, indicating that the bispecific antibody obtained in the present invention specifically bind to CLDN18.2.

### Example 3. In Vitro Functional Assay of Anti-CD40×CLDN18.2 Bispecific Antibody

In order to detect whether the bispecific antibody of the present invention can activate a NF-κB signaling pathway downstream of CD40 under different conditions, the example was detected through adopting a CD40-NF-κB-Jurkat luciferase reporter gene cell line stably expressing CD40 (Uniprot No. P25942).

### 3.1 Construction of CD40-NF-κB-Jurkat Luciferase Reporter Gene Cell Line

First, pGL4.30 plasmid (promega, #E8481) containing a NF-AT-re nucleic acid sequence was electroporated into Jurkat cells (ATCC®TIB-152™) through an electroporator (Invitrogen, Neon™ Transfection System, MP922947). The obtained cells were then transferred to RPMI 1640 medium (Hyclone, SH30243.01) containing 10% FBS (Gibco, 15140-141) by volume and without antibiotics. The cells were then inoculated into a 6-well cell culture dish and cultured for 48 h. The cells were then distributed into a 96-well cell culture plate at an average density of 1500 cells/well, and hygromycin B (Basalmedia, S160J7) was added at a final concentration of 500 μg/mL for screening. The growth of cell line clones was observed for about 2 to 3 weeks, and cloned cell lines were picked and transferred to a 24-well plate. After the cell culture was expanded, a part of the clones was transferred to a 96-well white-bottom plate (Corning, 3610) and stimulated with phorbol ester (using a concentration of 10 ng/ml) and ionomycin (using a concentration of 1 nM). After culturing in a 37° C. and 5% CO2 incubator for 6 h, Bright-Lite substrate (Vazyme, DD1204-03) was added. Expression levels of NF-κB in different clones were evaluated after reading signal values on a microplate reader (Molecular Devices: Spectramax i3x) to obtain a Jurkat cell line with high expression of NF-κB gene (named as NF-κB-Jurkat cells).

On this basis, a full-length expression gene sequence of human CD40 (Uniprot No. P25942) was stably transferred into the NF-κB-Jurkat cells to screen monoclonal cell lines. A CD40L recombinant protein (ACRO Biosystems, catalog number: CDL-H5248) was added to a cell line culture system. Intracellular NF-κB luciferase reporter gene transcription and expression were activated through a CD40-CD40L signal axis. The corresponding cell line expressing CD40 was detected and obtained through adding a catalytic substrate of luciferase to produce a fluorescent signal, named as a CD40-NF-κB-Jurkat luciferase reporter gene cell line.

### 3.2 Activation of NF-κB Signaling Pathway by Bispecific Antibody in Presence of Cross-Linking Agent

In this section, in the presence of a cross-linking agent, whether the binding of the bispecific antibody obtained in the present invention to CD40 has the ability to activate the downstream NF-κB signaling pathway was detected, wherein the expression of the luciferase reporter gene indicates the activation of the NF-κB signaling pathway.

The specific implementation is as follows. Serially diluted (28.9 nM in the first well and 5-fold serial dilution) bispecific antibody, positive control APX005, or isotype IgG1 negative control was mixed with the cross-linking agent (AffiniPure F(ab′)₂ Fragment Goat Anti-Human IgG, Fcγ fragment specific, Jackson Immunoresearch, catalog number: 109-006-098) at a final concentration of 5 μg/mL, plated in a 96-well cell culture plate, and cultured at room temperature for 30 min. Then, CD40-NF-κB-Jurkat cells were added to the cell culture plate at 1.0×10⁵ cells/well and cultured in a 37° C. incubator for 6 h. After culturing, 30 μL of luciferase substrate Bright-Lite (Vazyme, DD1204-03) was added to each well, and the fluorescence value of the 96-well plate was detected after shaking for 2 min.

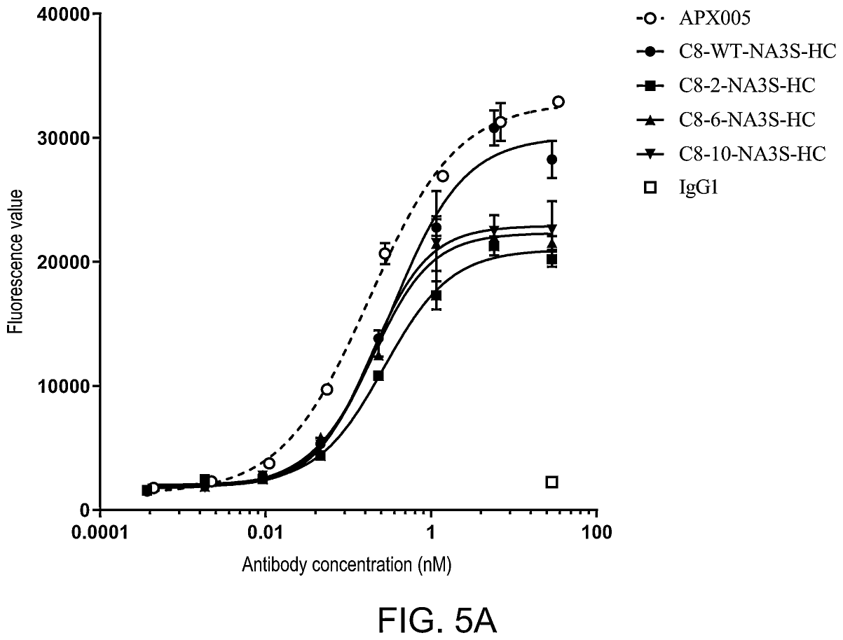

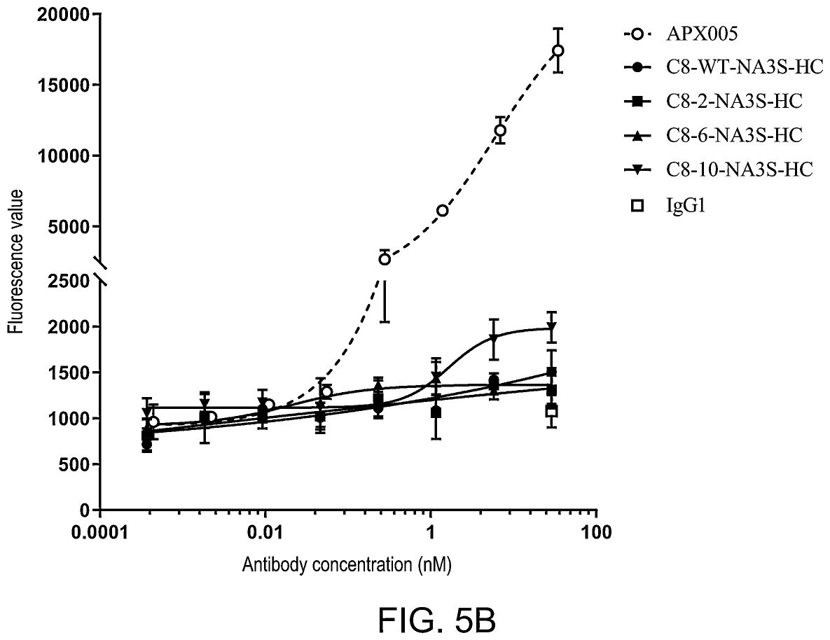

The results are shown in FIG. 5A. In the presence of the cross-linking agent, all the anti-CD40×CLDN18.2 bispecific antibodies underwent corresponding cross-linking reactions, which activated the NF-κB signaling pathway downstream of CD40 and produced fluorescence.

### 3.3 No Activation of NF-κB Signaling Pathway by Bispecific Antibody in Absence of Cross-Linking Agent

In this section, in the absence of a cross-linking agent, whether the binding of the bispecific antibody obtained in the present invention to CD40 has the ability to activate the downstream NF-κB signaling pathway was detected, wherein the expression of the luciferase reporter gene indicates the activation of the NF-κB signaling pathway.

The specific implementation was basically the same as the content disclosed in Section 3.2, except that no cross-linking agent was added.

The results are shown in FIG. 5B. In the absence of the cross-linking agent, the positive control APX005 activated the NF-κB signaling pathway downstream of CD40, while the bispecific antibody of the present invention basically did not or only slightly activated the NF-κB signaling pathway, and certain bispecific antibodies even performed comparable to the negative control. This indicates that in the absence of the cross-linking agent, the bispecific antibody of the present invention is a weak agonist antibody. Based on the weak activation ability of the bispecific antibody, it can be expected that the bispecific antibody of the present invention has less toxic side effects than the control antibody while weakly stimulating the CD40 molecules.

In the presence or the absence of the cross-linking effect, the bispecific antibody of the present invention exhibits obvious differences in agonistic activity, which is very effective in reducing non-specific agonistic activity and improving the therapeutic index.

### 3.4 Activation of NF-κB Signaling Pathway by Bispecific Antibody in Presence of 18.2-HEK293 Cells

In this section, in the presence of the 18.2-HEK293 cells, whether the binding of the bispecific antibody of the present invention to CD40 has the ability to activate the downstream NF-κB signaling pathway was detected, wherein the expression of the luciferase reporter gene indicates the activation of the NF-κB signaling pathway.

The specific implementation is as follows. The CD40-NF-κB-Jurkat cells were plated into a cell culture plate at 1.0×10⁵ cells/well, and the 18.2-HEK293 cells were then added at 1.0×10⁵ cells/well. Each serially diluted (5.8 nM in the first well, 10-fold dilution in the second well, 2-fold serial dilution in the third to seventh wells, and 4-fold dilution in the seventh to eighth wells) bispecific antibody or isotype IgG1 negative control was added to the plated cells and cultured in a 37° C. incubator for 6 h. After culturing, 30 μL of luciferase substrate Bright-Lite (Vazyme, DD1204-03) was added to each well, and the fluorescence value of the 96-well plate was detected after shaking for 2 min.

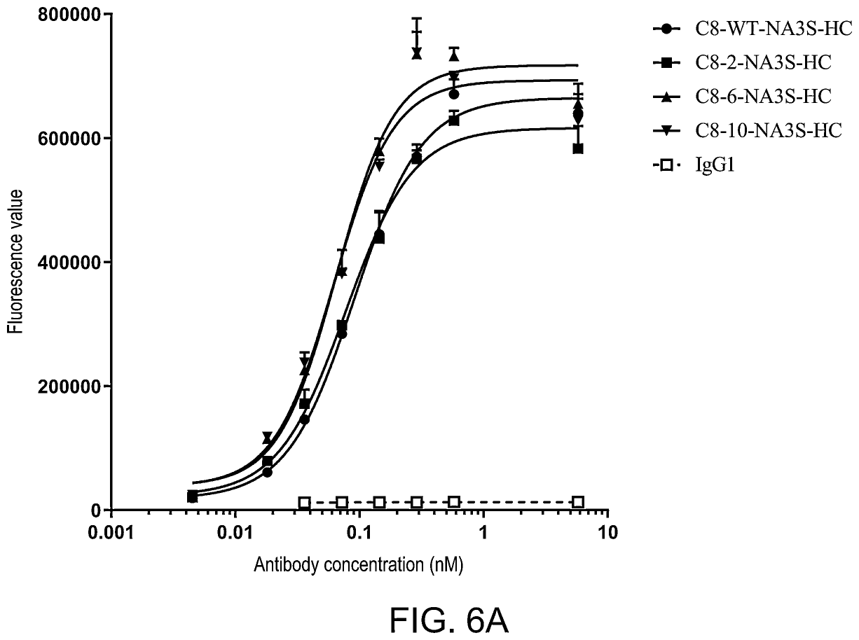

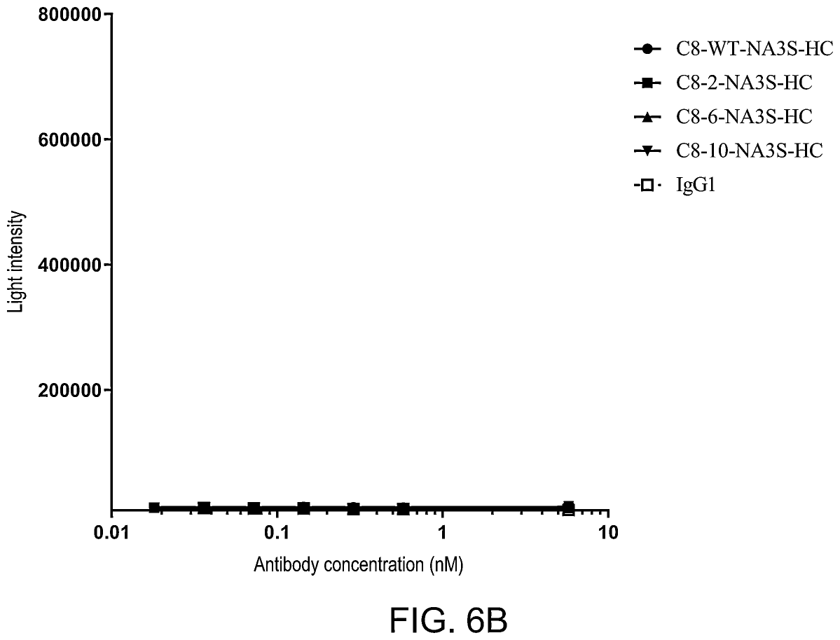

The results are shown in FIG. 6A. In the presence of the 18.2-HEK293 cells, each bispecific antibody of the present invention underwent a cross-linking effect, significantly activating the NF-κB signaling pathway downstream of CD40, and within a bispecific antibody concentration range of about 0.0045 nM to 5.8 nM, the activation ability was concentration-dependent.

### 3.5 No Activation of NF-κB Signaling Pathway by Bispecific Antibody in Presence of Negative Cells not Expressing CLDN18.2

In this section, in the presence of HEK293 cells not expressing CLDN18.2, whether the binding of the bispecific antibody of the present invention to CD40 has the ability to activate the downstream NF-κB signaling pathway was detected.

The specific implementation is as follows. The CD40-NF-κB-Jurkat cells were plated into a cell culture plate at 1.0×10⁵ cells/well, and HEK293 cells (ATCC®CRL-1573™) were then added at 1.0×10⁵ cells/well. Then, each diluted (5.8 nM in the first well, 10-fold dilution in the second well, 2-fold serial dilution in the third to seventh wells, and 4-fold dilution in the seventh to eighth wells) bispecific antibody or isotype IgG1 negative control was added to the plated cells and cultured in a 37° C. incubator for 6 h. After culturing, 30 μL of luciferase substrate Bright-Lite (Vazyme, DD1204-03) was added to each well, and the fluorescence value of the 96-well plate was detected after shaking for 2 min.

The results are shown in FIG. 6B. In the case of the HEK293 cells not expressing CLDN18.2, each bispecific antibody of the present invention did not activate the NF-κB signaling pathway downstream of CD40.

The results of FIGS. 6A and 6B can indicate that the ability of the bispecific antibody of the present invention to activate the NF-κB signaling pathway downstream of CD40 depends on the presence of CLDN18.2 and is concentration-dependent. This indicates that the bispecific antibody of the present invention forms a “cross-linking effect” through binding to CLDN18.2 on the 18.2-HEK293 cells, thereby effectively activating the NF-κB signaling pathway downstream of CD40 in the CD40-NF-κB-Jurkat cells, thereby promoting the immune response of APC cells and downstream T cells, achieving the effect of killing tumors. Furthermore, since the Fc region of the bispecific antibody is correspondingly modified, the non-specific binding of the antibody to hepatocytes is effectively prevented, thereby preventing or reducing the hepatotoxic side effects of the antibody.

### Example 4 Functional Assay of Anti-CD40×CLDN18.2 Bispecific Antibody in Animal Model

In the example, the tumor inhibition ability and the hepatotoxicity of the bispecific antibody of the present invention in an animal model were detected. In the example, tumor cells adopted were MC-38 cells (mouse colon cancer cells, Shanghai Model Organisms Center, catalog number: NM-S13-TM10) overexpressing CLDN18.2, hereinafter referred to as huCLDN18.2-MC38 cells (refer to Example 2.3 of the present application for the construction method). Experimental animals were 6 to 8 weeks old (20 to 22 g) female CD40 humanized mice C57BL/6-Cd40^{tm1 (CD40)}/Bcgen (Biocytogen, catalog number: 110009). The experimental mice were housed in independent ventilation boxes with constant temperature and humidity. In the breeding room, the temperature was 21 to 24° C. and the humidity was 30 to 53%. The resuspended huCLDN18.2-MC38 cells were subcutaneously injected into the right back of each mouse at a density of 2×10⁶ (day 0). When the subcutaneous tumor volume of the mice reached about 100 to 130 mm³ (day 7), mouse samples with large differences in tumor volumes were removed. Then, random grouping (5 mice in each group) into a PBS treatment group, a CCl₄ treatment group (chemical reagent hepatotoxicity positive control), a Selicrelumab monoclonal antibody administration group (Abgenix, clinical phase I, heavy chain and light chain amino acid sequences are respectively shown in SEQ ID NO:26 and SEQ ID NO:27), a C8-WT monoclonal antibody administration group (heavy chain amino acid sequence and light chain amino acid sequence respectively are shown in SEQ ID NO:28 and SEQ ID NO:23), and bispecific antibody C8-WT-NA3S-HC, C8-2-NA3S-HC administration groups was performed according to the tumor volumes. Each antibody administration group was set up with two dose groups of 1 mpk and 20 mpk. Administration was intravenous (iv) at twice a week for 3 weeks.

### 4.1 Detection of Tumor Inhibition Ability of Bispecific Antibody

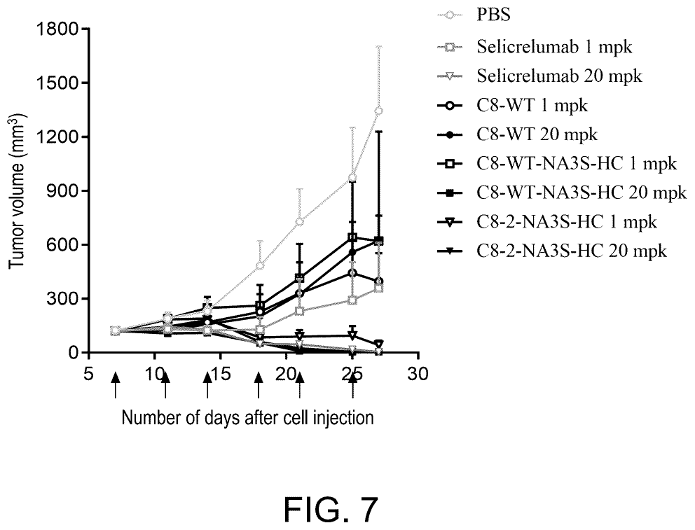

The length (mm) and the width (mm) of the tumor was observed and record at any time. The tumor volume (V) was calculated, the calculation manner was V=(length×width²)/2, and tumor growth inhibition rate TGI (%)=(1−average tumor volume of administration group/average tumor volume of PBS treatment group)×100%. The tumor inhibition results are shown in FIG. 7 and Tables 3 and 4. From the complete remission rate analysis results in Table 3, it can be seen that the bispecific antibodies C8-WT-NA3S-HC and C8-2-NA3S-HC showed complete tumor remission (tumor volume less than 5 mm³) at both high and low doses on day 18, while the control monoclonal antibody Selicrelumab only began to show complete tumor remission on day 21. All mice treated with the bispecific antibodies C8-WT-NA3S-HC and C8-2-NA3S-HC at a high dose showed complete tumor remission on day 25, while only 3 mice treated with the control monoclonal antibody Selicrelumab at a high dose showed complete remission on day 25. 2 mice died under the control monoclonal antibody Selicrelumab and the bispecific antibody C8-WT-NA3S-HC at a low dose, while no mice died under the bispecific antibody C8-2-NA3S-HC at both high and low doses. From the tumor inhibition results in FIG. 7 and Table 4, it can be seen that the bispecific antibodies C8-WT-NA3S-HC and C8-2-NA3S-HC have good tumor inhibition effects in mice, wherein the bispecific antibody C8-2-NA3S-HC has a significantly better tumor growth inhibition rate than the control monoclonal antibody Selicrelumab at a low dose. At the same time, the bispecific antibody displays a stronger tumor inhibition effect than the parent monoclonal antibody C8-WT, indicating that the bispecific antibody specifically kills CLDN18.2-positive tumor cells through cross-linking in vivo.

TABLE 3
Number of mice in complete remission of bispecific antibody

| | | | Number of mice in complete | | | | |
|---|---|---|---|---|---|---|---|
| | | | remission/Total number | | | | |
| Group | | | D 14 | D 18 | D 21 | D 25 | D 27 |
| Selicrelumab | 1 | mpk | 0/5 | 0/5 | 1/3 | 1/3 | 1/3 |
| | 20 | mpk | 0/5 | 0/5 | 0/5 | 3/5 | 5/5 |
| C8-WT | 1 | mpk | 0/5 | 0/5 | 0/5 | 0/5 | 0/3 |
| | 20 | mpk | 0/5 | 0/5 | 0/5 | 0/4 | 0/4 |
| C8-WT-NA3S-HC | 1 | mpk | 0/5 | 1/5 | 1/5 | 1/3 | 1/3 |
| | 20 | mpk | 0/5 | 2/5 | 4/5 | 5/5 | 5/5 |
| C8-2-NA3S-HC | 1 | mpk | 0/5 | 1/5 | 1/5 | 1/5 | 2/5 |
| | 20 | mpk | 0/5 | 1/5 | 3/5 | 5/5 | 5/5 |

TABLE 4
Tumor growth inhibition rate in mouse of bispecific antibody

| | Tumor growth inhibition rate (%) = (1 − Average tumor volume of | | | | | | | | |
|---|---|---|---|---|---|---|---|---|---|
| | experimental group/Average tumor volume of PBS control group) × 100% | | | | | | | | |
| | | | | | | C8-WT- | C8-WT- | C8-2- | C8-2- |
| Day of | | Selicrelumab | Selicrelumab | C8-WT | C8-WT | NA3S-HC | NA3S-HC | NA3S-HC | NA3S-HC |
| measurement | PBS | 1 mpk | 20 mpk | 1 mpk | 20 mpk | 1 mpk | 20 mpk | 1 mpk | 20 mpk |
| 11 | 0.0 | 27.93 | 11.65 | 25.07 | 24.00 | −4.26 | 41.06 | 19.85 | −9.43 |
| 14 | 0.0 | 40.39 | 34.62 | 26.81 | 19.44 | −10.75 | 49.59 | 19.51 | 2.57 |
| 18 | 0.0 | 68.33 | 88.10 | 54.73 | 51.37 | 48.22 | 87.80 | 83.34 | 83.98 |
| 21 | 0.0 | 77.56 | 92.64 | 58.35 | 48.49 | 45.49 | 98.53 | 88.07 | 95.99 |
| 25 | 0.0 | 73.58 | 97.34 | 44.48 | 33.05 | 19.81 | 99.43 | 87.15 | 99.40 |
| 27 | 0.0 | 76.49 | 99.55 | 72.63 | 46.54 | 63.97 | 99.59 | 95.69 | 99.57 |

### 4.2 Detection of Hepatotoxicity of Bispecific Antibody

The toxic side effects of drugs on the liver during administration are mainly judged through detecting levels of alanine aminotransferase (ALT) and aspartate aminotransferase (AST) in serum. The higher the levels, the greater the damage to hepatocytes. In the experiment, an alanine aminotransferase (glutamate pyruvate transaminase/ALT/GPT) test kit (Reitman method) microplate method (Nanjing Jiancheng Bioengineering Institute, catalog number: C009-2-1) and an aspartate aminotransferase (aspartate transferase/AST/GOT) test kit (microplate method) (Nanjing Jiancheng Bioengineering Institute, catalog number: C010-2-1) were adopted. The specific detection methods are as follows.

20 μL of matrix solution preheated at 37° C. was first added to assay wells of a 96-well plate, 5 μL of mouse serum collected at the end of administration was then added, and the mixture was gently shaken to mix. Only 20 μL of matrix solution preheated at 37° C. was added to control wells, and the mixture was gently shaken to mix. The 96-well plate was placed in a 37° C. constant temperature incubator for 30 min. Then, the 96-well plate was taken out. 20 μL of 2,4-dinitrophenylhydrazine solution was added to the assay wells, and the mixture was gently shaken to mix. 20 μL of 2,4-dinitrophenylhydrazine solution was first added to the control wells, 5 μL of distilled water was then added, and the mixture was gently shaken to mix for color development. Then, the 96-well plate was placed in a 37° C. constant temperature incubator for 20 min. Then, 200 μL of 0.4 mol/L NaOH was added to all the wells in the 96-well plate, and the mixture was gently shaken to stop the color development. Finally, after standing at room temperature for 10 min, the mixture was placed in a microplate reader for detection (wavelength λ=510 nm).

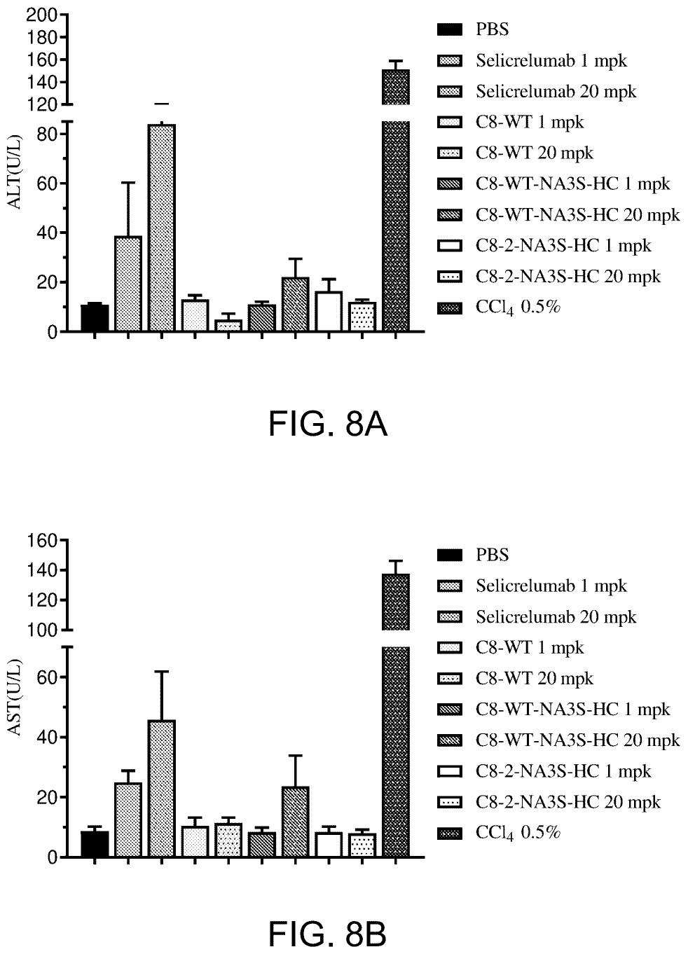

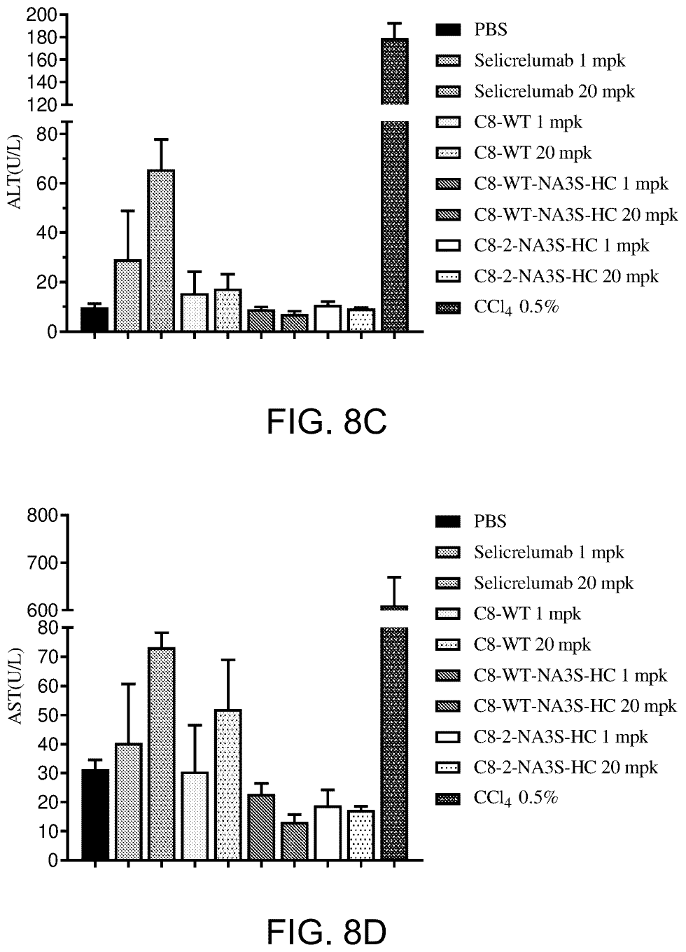

The blood of mice was respectively collected on day 21 and day 27 for detection of the levels of AST and ALT. The detection results of ALT and AST on day 21 are shown in FIGS. 8A and 8B, and the detection results of ALT and AST on day 27 are shown in FIGS. 8C and 8D. It can be seen that the values of ALT and AST of the bispecific antibody groups C8-WT-NA3S-HC and C8-2-NA3S-HC are equivalent to those of the PBS group, while the values of the Selicrelumab monoclonal antibody control group is 7 to 8 times that of the bispecific antibody group, indicating that the bispecific antibodies C8-WT-NA3S-HC and C8-2-NA3S-HC obtained in the present invention not only have better tumor inhibition effects and better targeting, but also have no toxic side effects on the liver, and are safer than Selicrelumab.

1. An anti-CD40×CLDN18.2 bispecific antibody, comprising (i) an anti-CD40 antibody or a fragment thereof and (ii) an anti-CLDN18.2 antibody or a fragment thereof, wherein:
the anti-CD40 antibody or the fragment thereof comprises 3 heavy chain CDRs contained in SEQ ID NO:11 or variants thereof and comprises 3 light chain CDRs contained in SEQ ID NO:10 or variants thereof;
the anti-CD40 antibody or the fragment thereof comprises 3 heavy chain CDRs contained in SEQ ID NO: 12 or variants thereof and comprises 3 light chain CDRs contained in SEQ ID NO:10 or variants thereof;
the anti-CD40 antibody or the fragment thereof comprises 3 heavy chain CDRs contained in SEQ ID NO:13 or variants thereof and comprises 3 light chain CDRs contained in SEQ ID NO: 10 or variants thereof; or
the anti-CD40 antibody or the fragment thereof comprises 3 heavy chain CDRs contained in SEQ ID NO: 14 or variants thereof and comprises 3 light chain CDRs contained in SEQ ID NO: 10 or variants thereof, wherein
the variants have one or two amino acid substitutions, additions, and/or deletions compared to sequences derived therefrom.

2. The bispecific antibody according to claim 1, wherein the anti-CLDN18.2 antibody or the fragment thereof is a single domain antibody (VHH), and the single domain antibody comprises 3 CDRs contained in SEQ ID NO:18 or variants thereof, wherein
the variants have one or two amino acid substitutions, additions, and/or deletions compared to sequences derived therefrom.

3. The bispecific antibody according to claim 1, comprising:
(i) a heavy chain with a structure VH-CH1-Fc-VHH, and
(ii) a light chain with a structure VL-CL, wherein
the VH comprises 3 heavy chain CDRs contained in SEQ ID NO:11, the VL comprises 3 light chain CDRs contained in SEQ ID NO:10, and the VHH comprises 3 CDRs contained in SEQ ID NO:18;
the VH comprises 3 heavy chain CDRs contained in SEQ ID NO: 12, the VL comprises 3 light chain CDRs contained in SEQ ID NO:10, and the VHH comprises 3 CDRs contained in SEQ ID NO:18;
the VH comprises 3 heavy chain CDRs contained in SEQ ID NO:13, the VL comprises 3 light chain CDRs contained in SEQ ID NO:10, and the VHH comprises 3 CDRs contained in SEQ ID NO:18; or
the VH comprises 3 heavy chain CDRs contained in SEQ ID NO:14, the VL comprises 3 light chain CDRs contained in SEQ ID NO:10, and the VHH comprises 3 CDRs contained in SEQ ID NO: 18.

4. The bispecific antibody according to claim 1, comprising:
(i) a heavy chain with a structure VH-CH1-Fc-VHH, and
(ii) a light chain with a structure VL-CL, wherein
the VH comprises 3 heavy chain CDRs shown in SEQ ID NO:4, SEQ ID NO:5, and SEQ ID NO:6, the VL comprises 3 light chain CDRs shown in SEQ ID NO:1, SEQ ID NO: 2, and SEQ ID NO:3, and the VHH comprises 3 CDRs shown in SEQ ID NO: 15, SEQ ID NO:16, and SEQ ID NO:17;
the VH comprises 3 heavy chain CDRs shown in SEQ ID NO:4, SEQ ID NO:5, and SEQ ID NO:7, the VL comprises 3 light chain CDRs shown in SEQ ID NO:1, SEQ ID NO: 2, and SEQ ID NO:3, and the VHH comprises 3 CDRs shown in SEQ ID NO: 15, SEQ ID NO:16, and SEQ ID NO:17;
the VH comprises 3 heavy chain CDRs shown in SEQ ID NO:4, SEQ ID NO:5, and SEQ ID NO:8, the VL comprises 3 light chain CDRs shown in SEQ ID NO:1, SEQ ID NO: 2, and SEQ ID NO:3, and the VHH comprises 3 CDRs shown in SEQ ID NO:15, SEQ ID NO:16, and SEQ ID NO:17; or
the VH comprises 3 heavy chain CDRs shown in SEQ ID NO:4, SEQ ID NO:5, and SEQ ID NO:9, the VL comprises 3 light chain CDRs shown in SEQ ID NO:1, SEQ ID NO: 2, and SEQ ID NO:3, and the VHH comprises 3 CDRs shown in SEQ ID NO:15, SEQ ID NO: 16, and SEQ ID NO: 17.

5. The bispecific antibody according to claim 1, wherein:
the VH comprises a sequence shown in SEQ ID NO:11 or a sequence having at least 90% identity with SEQ ID NO:11, the VL comprises a sequence shown in SEQ ID NO: 10 or a sequence having at least 90% sequence identity with SEQ ID NO:10, and the VHH comprises a sequence shown in SEQ ID NO: 18 or a sequence having at least 90% sequence identity with SEQ ID NO:18;
the VH comprises a sequence shown in SEQ ID NO: 12 or a sequence having at least 90% identity with SEQ ID NO: 12, the VL comprises a sequence shown in SEQ ID NO: 10 or a sequence having at least 90% sequence identity with SEQ ID NO:10, and the VHH comprises a sequence shown in SEQ ID NO:18 or a sequence having at least 90% sequence identity with SEQ ID NO:18;
the VH comprises a sequence shown in SEQ ID NO:13 or a sequence having at least 90% identity with SEQ ID NO:13, the VL comprises a sequence shown in SEQ ID NO: 10 or a sequence having at least 90% identity with SEQ ID NO: 10, and the VHH comprises a sequence shown in SEQ ID NO:18 or a sequence having at least 90% identity with SEQ ID NO:18; or
the VH comprises a sequence shown in SEQ ID NO: 14 or a sequence having at least 90% identity with SEQ ID NO:14, the VL comprises a sequence shown in SEQ ID NO: 10 or a sequence having at least 90% sequence identity with SEQ ID NO: 10, and the VHH comprises a sequence shown in SEQ ID NO: 18 or a sequence having at least 90% sequence identity with SEQ ID NO:18.

6. The bispecific antibody according to claim 1, wherein the VHH is connected to Fc through a linker, preferably, the linker is (G₄S) n, where n is an integer equal to or greater than 1, for example, n is an integer of 1, 2, 3, 4, 5, 6, 7, 8, or 9, more preferably, the linker is selected from (G₄S)₃.

7. The bispecific antibody according to claim 1, wherein an Fc region is an Fc region of an IgG class antibody, such as an Fc region of IgG1, such as an Fc region of a modified IgG1, such as an Fc region of IgG1 with an N297Q mutation.

8. The bispecific antibody according to claim 1, wherein a light chain constant domain CL of the antibody is from κ or λ.

9. The bispecific antibody according to claim 1, comprising:
(i) a heavy chain with a structure VH-CH1-Fc-VHH, and
(ii) a light chain with a structure VL-CL, wherein
the heavy chain comprises a sequence shown in SEQ ID NO: 19 or a sequence at least 90% identical to SEQ ID NO:19, and the light chain comprises a sequence shown in SEQ ID NO:23 or a sequence at least 90% identical to SEQ ID NO:23;
the heavy chain comprises a sequence shown in SEQ ID NO:20 or a sequence at least 90% identical to SEQ ID NO:20, and the light chain comprises a sequence shown in SEQ ID NO:23 or a sequence at least 90% identical to SEQ ID NO:23;
the heavy chain comprises a sequence shown in SEQ ID NO:21 or a sequence at least 90% identical to SEQ ID NO:21, and the light chain comprises a sequence shown in SEQ ID NO:23 or a sequence at least 90% identical to SEQ ID NO:23; or
the heavy chain comprises a sequence shown in SEQ ID NO:22 or a sequence at least 90% identical to SEQ ID NO:22, and the light chain comprises a sequence shown in SEQ ID NO:23 or a sequence at least 90% identical to SEQ ID NO:23.

10. A polynucleotide encoding the bispecific antibody according to claim 1.

11. A vector comprising the polynucleotide according to claim 10, preferably being an expression vector.

12. A host cell comprising the polynucleotide according to claim 10.

13. A method for producing the bispecific antibody according to claim 1, comprising steps of (i) culturing the host cell of the present invention under conditions suitable for expressing the bispecific antibody of the present invention, and optionally, (ii) recovering the bispecific antibody of the present invention.

14. An immunoconjugate, a kit, a pharmaceutical composition, a combination product, or a preparation comprising the bispecific antibody according to claim 1.

15. The kit, the pharmaceutical composition, the combination product, or the preparation according to claim 14, further comprising other therapeutic agents and optional pharmaceutical aids, wherein preferably, the other therapeutic agents are selected from chemotherapeutic agents and cytotoxic agents.

16. A use of the bispecific antibody according to claim 1 in a medicament or a kit for treating, preventing, and/or diagnosing a disease related to CD40 or a disease related to CLDN18.2, wherein preferably, the disease related to CLDN18.2 is a cancer in which CLDN18.2 is aberrantly expressed, and the disease related to CD40 is a cancer in which CD40 is aberrantly expressed.

17. A method for treating a disease related to CD40 or a disease related to CLDN18.2, comprising administering a therapeutically effective amount of the bispecific antibody according to claim 1 to a patient in need.


SEQUENCE LISTING (28 sequences)

SEQ ID NO: 1 - protein, 11 aa, synthetic construct
  REGION 1..11: note = C8-WT LCDR1
RSSQGIRSSLA

SEQ ID NO: 2 - protein, 7 aa, synthetic construct
  REGION 1..7: note = C8-WT LCDR2
GASSLEG

SEQ ID NO: 3 - protein, 9 aa, synthetic construct
  REGION 1..9: note = C8-WT LCDR3
QQLNSFPST

SEQ ID NO: 4 - protein, 10 aa, synthetic construct
  REGION 1..10: note = C8-WT HCDR1
GFTFGSYEMN

SEQ ID NO: 5 - protein, 10 aa, synthetic construct
  REGION 1..10: note = C8-WT HCDR2
YISSSGETTD

SEQ ID NO: 6 - protein, 18 aa, synthetic construct
  REGION 1..18: note = C8-WT HCDR3
DVFFFDSSGDNGAYGMDV

SEQ ID NO: 7 - protein, 18 aa, synthetic construct
  REGION 1..18: note = C8-2 HCDR3
DVFFFSPSGDNGAYGMDV

SEQ ID NO: 8 - protein, 18 aa, synthetic construct
  REGION 1..18: note = C8-6 HCDR3
DVFFFDSSGSPGAYGMDV

SEQ ID NO: 9 - protein, 18 aa, synthetic construct
  REGION 1..18: note = C8-10 HCDR3
DVFFFDSSGFPGAYGMDV

SEQ ID NO: 10 - protein, 107 aa, synthetic construct
  REGION 1..107: note = C8-WT VL
DIQLTQSPSSLTASVGDRVTITCRSSQGIRSSLAWYQQKPGKPPKLLIYGASSLEGGVPSRFSGSGSGTDFTLTISSLQPEDFATYYCQQLNSFPSTFGQGTKLEIK

SEQ ID NO: 11 - protein, 127 aa, synthetic construct
  REGION 1..127: note = C8-WT VH
EVQLLESGGGLVRPGGSLRLSCAASGFTFGSYEMNWVRQAPGKGLEWVSYISSSGETTDYADSVKGRFTVSRDNSKNLLYLQMNSLRAEDTAVYYCARDVFFFDSSGDNGAYGMDVWGQGTMVTVSS

SEQ ID NO: 12 - protein, 127 aa, synthetic construct
  REGION 1..127: note = C8-2 VH
EVQLLESGGGLVRPGGSLRLSCAASGFTFGSYEMNWVRQAPGKGLEWVSYISSSGETTDYADSVKGRFTVSRDNSKNLLYLQMNSLRAEDTAVYYCARDVFFFSPSGDNGAYGMDVWGQGTMVTVSS

SEQ ID NO: 13 - protein, 127 aa, synthetic construct
  REGION 1..127: note = C8-6 VH
EVQLLESGGGLVRPGGSLRLSCAASGFTFGSYEMNWVRQAPGKGLEWVSYISSSGETTDYADSVKGRFTVSRDNSKNLLYLQMNSLRAEDTAVYYCARDVFFFDSSGSPGAYGMDVWGQGTMVTVSS

SEQ ID NO: 14 - protein, 127 aa, synthetic construct
  REGION 1..127: note = C8-10 VH
EVQLLESGGGLVRPGGSLRLSCAASGFTFGSYEMNWVRQAPGKGLEWVSYISSSGETTDYADSVKGRFTVSRDNSKNLLYLQMNSLRAEDTAVYYCARDVFFFDSSGFPGAYGMDVWGQGTMVTVSS

SEQ ID NO: 15 - protein, 10 aa, synthetic construct
  REGION 1..10: note = NA3SH1-T4-hVH6 CDR1
GSIFHIPVMG

SEQ ID NO: 16 - protein, 9 aa, synthetic construct
  REGION 1..9: note = NA3SH1-T4-hVH6 CDR2
GISRGGTTN

SEQ ID NO: 17 - protein, 12 aa, synthetic construct
  REGION 1..12: note = NA3SH1-T4-hVH6 CDR3
LVVSGIGSTLEV

SEQ ID NO: 18 - protein, 120 aa, synthetic construct
  REGION 1..120: note = NA3SH1-T4-hVH6 VHH
QVQLVESGGGLVQPGGSLRLSCAASGSIFHIPVMGWYRQAPGKGLELVAGISRGGTTNYADSVKGRFTISRDNAKNTLYLQMNSLRAEDTAVYYCNVLVVSGIGSTLEVWGQGTLVTVSS

SEQ ID NO: 19 - protein, 592 aa, synthetic construct
  REGION 1..592: note = C8-WT-NA3S-HC HC
EVQLLESGGGLVRPGGSLRLSCAASGFTFGSYEMNWVRQAPGKGLEWVSYISSSGETTDYADSVKGRFTVSRDNSKNLLYLQMNSLRAEDTAVYYCARDVFFFDSSGDNGAYGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVESGGGLVQPGGSLRLSCAASGSIFHIPVMGWYRQAPGKGLELVAGISRGGTTNYADSVKGRFTISRDNAKNTLYLQMNSLRAEDTAVYYCNVLVVSGIGSTLEVWGQGTLVTVSS

SEQ ID NO: 20 - protein, 592 aa, synthetic construct
  REGION 1..592: note = C8-2-NA3S-HC HC
EVQLLESGGGLVRPGGSLRLSCAASGFTFGSYEMNWVRQAPGKGLEWVSYISSSGETTDYADSVKGRFTVSRDNSKNLLYLQMNSLRAEDTAVYYCARDVFFFSPSGDNGAYGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVESGGGLVQPGGSLRLSCAASGSIFHIPVMGWYRQAPGKGLELVAGISRGGTTNYADSVKGRFTISRDNAKNTLYLQMNSLRAEDTAVYYCNVLVVSGIGSTLEVWGQGTLVTVSS

SEQ ID NO: 21 - protein, 592 aa, synthetic construct
  REGION 1..592: note = C8-6-NA3S-HC HC
EVQLLESGGGLVRPGGSLRLSCAASGFTFGSYEMNWVRQAPGKGLEWVSYISSSGETTDYADSVKGRFTVSRDNSKNLLYLQMNSLRAEDTAVYYCARDVFFFDSSGSPGAYGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVESGGGLVQPGGSLRLSCAASGSIFHIPVMGWYRQAPGKGLELVAGISRGGTTNYADSVKGRFTISRDNAKNTLYLQMNSLRAEDTAVYYCNVLVVSGIGSTLEVWGQGTLVTVSS

SEQ ID NO: 22 - protein, 592 aa, synthetic construct
  REGION 1..592: note = C8-10-NA3S-HC HC
EVQLLESGGGLVRPGGSLRLSCAASGFTFGSYEMNWVRQAPGKGLEWVSYISSSGETTDYADSVKGRFTVSRDNSKNLLYLQMNSLRAEDTAVYYCARDVFFFDSSGFPGAYGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVESGGGLVQPGGSLRLSCAASGSIFHIPVMGWYRQAPGKGLELVAGISRGGTTNYADSVKGRFTISRDNAKNTLYLQMNSLRAEDTAVYYCNVLVVSGIGSTLEVWGQGTLVTVSS

SEQ ID NO: 23 - protein, 214 aa, synthetic construct
  REGION 1..214: note = C8-WT-NA3S-HC LC
DIQLTQSPSSLTASVGDRVTITCRSSQGIRSSLAWYQQKPGKPPKLLIYGASSLEGGVPSRFSGSGSGTDFTLTISSLQPEDFATYYCQQLNSFPSTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 352 aa, synthetic construct
  REGION 1..352: note = NA3SH1-T4-Hvh6
QVQLVESGGGLVQPGGSLRLSCAASGSIFHIPVMGWYRQAPGKGLELVAGISRGGTTNYADSVKGRFTISRDNAKNTLYLQMNSLRAEDTAVYYCNVLVVSGIGSTLEVWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 15 aa, synthetic construct
  REGION 1..15: note = linker
GGGGSGGGGSGGGGS

SEQ ID NO: 26 - protein, 452 aa, synthetic construct
  REGION 1..452: note = Selicrelumab HC
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 27 - protein, 214 aa, synthetic construct
  REGION 1..214: note = Selicrelumab LC
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 457 aa, synthetic construct
  REGION 1..457: note = C8-WT HC
EVQLLESGGGLVRPGGSLRLSCAASGFTFGSYEMNWVRQAPGKGLEWVSYISSSGETTDYADSVKGRFTVSRDNSKNLLYLQMNSLRAEDTAVYYCARDVFFFDSSGDNGAYGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK